(19) United States
(12) Patent Application Publication
(10) Pub. No.: US 2025/0135235 A1
(43) Pub. Date: May 1, 2025

(54) INTRA-OPERATIVE RADIATION DOSE FEEDBACK SYSTEM

(71) Applicant: GT MEDICAL TECHNOLOGIES, INC., Tempe, AZ (US)

(72) Inventors: David Brachman, Phoenix, AZ (US); Heidi Cole, Phoenix, AZ (US); John Baker, Gilbert, AZ (US); Adam Turner, Phoenix, AZ (US)

(21) Appl. No.: 18/836,571

(22) PCT Filed: Feb. 23, 2023
(86) PCT No.: PCT/US2023/063174

§ 371 Date: Aug. 7, 2024

Related U.S. Application Data
(60) Provisional application No. 63/314,053, filed on Feb. 25, 2022.

(51) Int. Cl. A61N 5/10

(52) U.S. Cl. CPC A61N 5/1071; A61N 5/1007

(57) ABSTRACT

A dose feedback system provides real-time or near real-time intra-operative radiation dose feedback system to improve and/or ensure that a proper amount of radiation is delivered to a patient by, for example, carriers (e.g., tile carriers). The dose feedback system can be implemented during medical surgeries, operations, procedures, etc. The dose feedback system may allow a medical professional (e.g., surgeon) to more accurately achieve a prescribed radiation of a dosimetric intent or a more detailed dosimetric plan through adjustments to position, dose, quantity, etc. of carriers prior to terminating the medical procedure (e.g., cavity of tumor bed is closed and/or patient is brought out from under anesthesia) at which point adjusting the radiation dose may be difficult or impossible.

### FIELD

The present disclosure generally relates to devices used in conjunction with radiation therapy.

### BACKGROUND

Tumors in living organisms are highly variable in size, location and their amount of infiltration into normal tissues, the variability of tumors in general make them very difficult to treat with a one-size fits all approach. Furthermore, the extent of tumors and/or void upon debulking are typically not known until presented in the operating room. Thus, the options necessary to effectively treat a tumor or tumor bed need to be quite diverse.

Brachytherapy involves placing a radiation source either into or immediately adjacent to a tumor. It provides an effective treatment of cancers of many body sites. Brachytherapy, as a component of multimodality cancer care, provides cost-effective treatment. Brachytherapy may be intracavitary, such as when treating gynecologic malignancies; intraluminal, such as when treating esophageal or lung cancers; external surface, such as when treating cancers of the skin, or interstitial, such as when treating various central nervous system tumors as well as extracranial tumors of the head and neck, lung, soft tissue, gynecologic sites, rectum, liver, prostate, penis and skin.

### SUMMARY

Various embodiments of systems, methods and devices within the scope of the appended claims each have several aspects, no single one of which is solely responsible for the desirable attributes described herein. Without limiting the scope of the appended claims, the description below describes some prominent features.

Details of one or more embodiments of the subject matter described in this specification are set forth in the accompanying drawings and the description below. Other features, aspects, and advantages will become apparent from the description, the drawings, and the claims. Note that relative dimensions of the following figures may not be drawn to scale.

Various features of these improvements are discussed below, and may be used together in various combinations or independently. For example, the implant placement system (also referred to herein as simply “the system”) could be used to aid a surgeon in placing physical implants based on an implant plan developed by an implant planning system, such as software that determines a radiation treatment plan for a patient based on a prescribed radiation dose.

While the examples shown use radioactive implants placed on the brain for the purposes of treating cancer, the systems and methods described herein may be used in many other applications.

In the examples shown, a photographic image of the brain is used for the purposes of planning implant placement. However, the planning image may be any two-dimensional (2D) image or three-dimensional (3D) surface, for example created using 3D surface rendering of information acquired using Magnetic resonance imaging (MRI), computed tomography (CT), ultrasound, or optical imaging.

In some aspects, the techniques described herein relate to a radiation dose feedback system including: one or more sensors including at least a camera positioned to generate a video stream of a treatment surface of a patient; a display device positioned in an implant placement room to be viewed by a user; one or more hardware processors configured to execute program instructions to cause the system to, for each of one or more proxy carriers positioned on the treatment surface: determine placement information of the proxy carrier based at least on sensor data from the one or more sensors, wherein the placement information indicates a three-dimensional position and orientation of the proxy carrier with reference to a fixed origin point; and determine carrier characteristics associated with the proxy carrier, the carrier characteristics indicating at least a radiation dose of a hot carrier represented by the proxy carrier, wherein the proxy carrier does not include a radiation source; determine, based on the determined placement information and carrier characteristics of each of the one or more proxy carriers, an expected dosimetric distribution; and display, on the display device, the video stream of the treatment surface overlaid with the expected dosimetric distribution.

In some aspects, the techniques described herein relate to a system, wherein the one or more sensors include a probe including internal sensors configured to determine a three dimensional position of a tip of the probe with reference to the fixed origin point.

In some aspects, the techniques described herein relate to a system, wherein placement information of a first proxy carrier is determined by: touching the probe tip to each of one or more markings on the first proxy carrier; determining placement information of the first proxy carrier based at least on respective position of the probe tip while the probe tip touches respective marking.

In some aspects, the techniques described herein relate to a system, wherein the first proxy carrier includes two or more markings at known locations on a surface of the first proxy carrier.

In some aspects, the techniques described herein relate to a system, wherein the placement information for each proxy carrier includes two or more three-dimensional coordinates each indicating an x, y, and z distance from the fixed origin point.

In some aspects, the techniques described herein relate to a system, wherein the sensor data used to determine location information of a first proxy carrier includes the video stream from the camera, wherein the hardware processor is configured to detect one or more markings of the proxy carrier within the video stream.

In some aspects, the techniques described herein relate to a system, wherein an orientation of the first proxy carrier is determined based on one or more of a size or a shape of the one or more markings.

In some aspects, the techniques described herein relate to a system, wherein a laser sensor is configured to determine a distance from the laser to each of the one or more markings of the proxy carrier, which is usable to determine location and/or orientation of the proxy carrier.

In some aspects, the techniques described herein relate to a system, wherein an infrared sensor is configured to determine a distance from the infrared sensor to each of the one or more markings of the proxy carrier, which is usable to determine location and/or orientation of the proxy carrier.

In some aspects, the techniques described herein relate to a system, wherein a distance to each of the one or more markings of the proxy carrier is determined based at least on the video data.

In some aspects, the techniques described herein relate to a system, wherein the proxy carriers include one or more of the sensors and placement information of the proxy carriers is determined based on sensor data received from the one or more proxy carriers.

In some aspects, the techniques described herein relate to a radiation dose feedback system including: one or more sensors including at least a camera positioned to generate a video stream of a treatment surface of a patient; a display device positioned in an implant placement room to be viewed by a user; one or more hardware processors configured to execute program instructions to cause the system to, for each of one or more carriers positioned on the treatment surface: determine placement information of the carrier based at least on sensor data from the one or more sensors, wherein the placement information indicates a three-dimensional position and orientation of the carrier with reference to a fixed origin point; and determine carrier characteristics associated with the carrier, the carrier characteristics including at least a radiation dose; determine, based on the determined placement information and carrier characteristics of each of the one or more carriers, an expected dosimetric distribution; and display, on the display device, the video stream of the treatment surface overlaid with the expected dosimetric distribution.

In some aspects, the techniques described herein relate to a system, wherein the carriers each include one or more radiation source.

In some aspects, the techniques described herein relate to a system, wherein the carriers are proxy carriers that do not include a radiation source.

In some aspects, the techniques described herein relate to a system, wherein the one or more sensors include a probe including internal sensors configured to determine a three dimensional position of a tip of the probe with reference to the fixed origin point.

In some aspects, the techniques described herein relate to a system, wherein placement information of a first carrier is determined by: touching the probe tip to each of one or more markings on the first carrier; and determining placement information of the first carrier based at least on respective position of the probe tip while the probe tip touches respective marking.

In some aspects, the techniques described herein relate to a radiation dose feedback system including: one or more sensors configured to obtain data relating to a placement position or orientation of one or more proxy carriers with respect to a treatment area of a patient; one or more surgical cameras configured to capture image or video data of the treatment area; a feedback device configured to display one or more images or videos; and one or more hardware processors in communication with the one or more sensors and the feedback device, wherein the one or more hardware processors are configured to execute program instructions to cause the system to: generate deformation data of the tumor bed of the patient, wherein the deformation data is based, at least in part, on one or more images of the tumor bed and a deformation analysis of the tumor bed; receive information from the one or more sensors including the data relating to a placement position or orientation of the one or more proxy carriers with respect to the tumor bed; determine an expected dosimetric distribution, wherein the expected dosimetric distribution is based, at least in part, on the data relating to a placement position or orientation of the one or more proxy carriers and the deformation data; receive, from the one or more surgical cameras, image or video data of the tumor bed; display, via the feedback device, one or more images or videos of the tumor bed based on the received image or video data of the tumor bed; and display, via the feedback device, a visual representation of the expected dosimetric distribution, wherein the visual representation is displayed as superimposed on the displayed on the one or more images or videos of the tumor bed.

In some aspects, the techniques described herein relate to a radiation dose feedback system, wherein the one or more hardware processors are configured to execute program instructions to cause the system to: receive carrier characteristics associated with the one or more proxy carriers, wherein the carrier characteristics include: an identification of the one or more proxy carriers; a magnitude of radiation expected to emit from one or more carriers to be placed in the tumor bed; and a direction of radiation expected to emit from the one or more carriers.

In some aspects, the techniques described herein relate to a radiation dose feedback system, wherein the one or more hardware processors are configured to execute program instructions to cause the system to: determine the expected dosimetric distribution, based at least in part, on the carrier characteristics.

In some aspects, the techniques described herein relate to a radiation dose feedback system, wherein the one or more hardware processors are configured to execute program instructions to cause the system to: display, via the feedback device, a depth visual indicator to indicate a desired distance from the tumor bed at which a certain radiation dose is desired to be delivered.

In some aspects, the techniques described herein relate to a radiation dose feedback system, wherein the one or more hardware processors are configured to execute program instructions to cause the system to: display, via the feedback device, a placement visual indicator to indicate the placement position or orientation of the one or more proxy carriers in the tumor bed.

In some aspects, the techniques described herein relate to a radiation dose feedback system, wherein the one or more hardware processors are configured to execute program instructions to cause the system to: determine whether the expected dosimetric distribution is acceptable; and in response to determining, that the expected dosimetric distribution is acceptable, output, via the feedback device, one or more auditory or visual signals that the expected dosimetric distribution is acceptable.

In some aspects, the techniques described herein relate to a radiation dose feedback system, wherein the one or more hardware processors are configured to execute program instructions to cause the system to: determine whether the expected dosimetric distribution is acceptable based at least in part on a degree to which the expected dosimetric distribution matches a dosimetric intent.

In some aspects, the techniques described herein relate to a radiation dose feedback system, wherein the one or more hardware processors are configured to execute program instructions to cause the system to: in response to determining, that the expected dosimetric distribution is not acceptable, output, via the feedback device, one or more auditory or visual signals that the expected dosimetric distribution is not acceptable.

In some aspects, the techniques described herein relate to a radiation dose feedback system, wherein feedback device includes a display screen.

In some aspects, the techniques described herein relate to a radiation dose feedback system, wherein the feedback device includes virtual reality (VR) or augmented reality (AR) systems or devices.

In some aspects, the techniques described herein relate to a radiation dose feedback system, wherein the feedback device is configured to display the one or more images or videos as three dimensional.

In some aspects, the techniques described herein relate to a radiation dose feedback system, wherein the one or more hardware processors are configured to execute program instructions to cause the system to: display, via the feedback device, the one or more images or videos of the tumor bed as three dimensional; display, via the feedback device, the visual representation of the expected dosimetric distribution as three dimensional, wherein the visual representation is displayed as within the three dimensions of the one or more images or videos of the tumor bed.

In some aspects, the techniques described herein relate to a radiation dose feedback system, wherein the one or more sensors is a probe including a tip portion, wherein the probe is configured to: contact the one or more proxy carriers via the tip portion; generate the data relating to the placement position or orientation of the one or more proxy carriers in response to contacting the one or more proxy carriers via the tip portion; and transmit, to one or more hardware processors, the data relating to the placement position or orientation of the one or more proxy carriers.

In some aspects, the techniques described herein relate to a radiation dose feedback system, wherein the one or more sensors is included as part of the one or more proxy carriers.

### BRIEF DESCRIPTION OF THE DRAWINGS

FIG. 1A is a block diagram illustrating an example real-time intra-operative radiation dose feedback system.

FIG. 1B-1C illustrates example implementations of a real-time intra-operative radiation dose feedback system during a medical procedure.

FIG. 1D is a block diagram illustrating an example placement software module including various modules for controlling operation of an implant placement system.

FIG. 2A is a block diagram illustrating an example proxy carrier with various components.

FIGS. 2B-2D are perspective views of example proxy carriers.

FIGS. 3A-3B illustrate a tumor bed of a patient with carriers placed therein.

FIGS. 4A-4G illustrate example displays of a feedback device of a real-time intra-operative radiation dose feedback system.

FIG. 5 illustrates an example image of a postoperative surgical site with carriers implanted therein.

FIG. 6 is a flowchart illustrating an example process for placing hot carriers using intra-operative dose feedback using proxy carriers.

FIG. 7 is a flowchart illustrating an example process of using proxy carriers to intra-operatively determine hot carrier placement.

### DETAILED DESCRIPTION

### Overview

Tumors are difficult to eradicate surgically as their infiltrative nature often precludes microscopically complete resection without undue morbidity or mortality. This local persistence of tumor cells may be controlled if sufficient radiation can be delivered safely prior to regrowth and replication of the residual tumor cells. Debulking surgery, followed by radiation therapy may be used for local control of a tumor. Discussed herein are various systems, methods, and devices for use in conjunction with radiation therapy, such as to deliver (and to control delivery of) radiation to a post-operative tumor bed.

### Terms

To facilitate an understanding of the systems and methods discussed herein, several terms are described below. These terms, as well as other terms used herein, should be construed to include the provided descriptions, the ordinary and customary meanings of the terms, and/or any other implied meaning for the respective terms, wherein such construction is consistent with context of the term. Thus, the descriptions below do not limit the meaning of these terms, but only provide example descriptions.

Tumor: an abnormal growth of tissue resulting from uncontrolled, progressive multiplication of cells. Tumors can be benign or malignant.

Tumor bed: an anatomical area of a patient (e.g., a human or other mammal) where a tumor exists (pre-operative tumor bed) and/or an area surrounding a surgically removed tumor (e.g., a resection cavity or post-operative tumor bed), such as a cranial cavity from which a tumor was surgically removed. Even after surgical removal of a tumor, the remaining tumor bed of the patient may include tumor cells.

Treatment area: an anatomical area that is targeted for delivery of radiation, such as from one or more radiation delivery devices (e.g., the carriers discussed below). A treatment area may include tissue below and/or around a location where the radiation delivery device is positioned, such as an anatomical area of a tumor or a tumor bed.

Treatment surface: an anatomical surface of a patient (e.g., a human or other mammal) where a radiation delivery device is to be placed to deliver radiation to a treatment area, such as the treatment surface itself and/or tissue below the treatment surface. A treatment surface may be a portion of a tumor bed or any other anatomical surface. For example, if a tumor bed is surgically created, the treatment surface may include an entire exposed surface of the tumor bed, a portion of such exposed surface, or the entire exposed surface of the tumor bed as well as a surrounding area of tissue.

Radiation Source: a radioactive material that is configured for delivery of radiation to a tumor and/or tumor bed. A radiation source may be in various shapes and sizes, such as cylinder, cone, sphere, pyramid, cube, prism, rectangular prism, triangular prism, and/or any combination of these or other shapes. One type of radiation source is a “Radioactive Seed” or simply “Seed.” While seeds are generally referred to herein as cylindrical, any other shape or size of see or other radioactive source may alternatively be used in the various systems and methods discussed herein. Seeds may comprise any combination of one or more of multiple radioactive components, such as Cs 131, Ir 192, I 125, Pd 103, for example. Seeds may include a protective outer shell that partially or fully encases the radioactive material. Seeds are one form of radiation source. The term “radiation source,” as used herein, may also refer to a radioactive seed (or other object that emits radiation) that is embedded, or otherwise attached to, a carrier (e.g., a tile carrier with an embedded radioactive seed).

Brachytherapy: radiation treatment in which the radiation delivery device (e.g., a radiation source) is placed directly on and/or close to a treatment surface of the body, such as directly on the surface of the body, within the body, or in a tumor bed. For example, brachytherapy may be intracavitary, such as in cranial or gynecologic malignancies; intraluminal, such as in esophageal or lung cancers; external, such as in cancers of the skin; and/or interstitial, such as in treatment of various central nervous system tumors as well as extracranial tumors of the head, neck, lung, soft tissue, gynecologic sites, rectum, liver, prostate, and penis.

Therapeutic Index: relationship between an amount of therapeutic effect provided by a therapeutic agent, such as one or more radioactive seeds in carriers, to an amount that causes toxicity. The therapeutic index may indicate a relative amount of healthy tissue (non-target tissue) receiving radiation (e.g., above a certain dosage level) compared to an amount of the target area (e.g., a tumor or tumor bed) receiving radiation. The therapeutic index may be a ratio of radiation delivered to a treatment area (e.g., tumor or tumor bed) to radiation delivered to areas surrounding the treatment area. Thus, a higher therapeutic index generally indicates better localization of radiation to the treatment area, sparing as much of the surrounding area from radiation as possible. Accordingly, improving the therapeutic index may increase local control of tumors and/or decrease the morbidity of treatment.

Implant: A device that is placed in or on the patient (e.g., human or animal) for the purpose of treating the patient. Implants may emit various types of energy or chemical agents. For example, implants may contain radioactive material that emits radiation for the purpose of treating tumors. In another example, implants may emit chemicals, such as chemotherapeutic agents used to treat cancer or other agents used, for example, to promote soft tissue or bone healing or regeneration. For ease of description, examples herein are primarily discussed with reference to implants comprising a carrier that contains one or more radioactive seeds. However, any other implants are compatible with the systems and methods discussed herein.

Carrier: substrate that holds or contains one or more radioactive seed. A carrier that contains one or more seeds is a radiation delivery device. Carriers may comprise various materials, such as one or more biocompatible and/or bioresorbable materials, such as collagen. Thus, these bioresorbable materials are biodegradable, or naturally absorbing into the mammalian tissue over time, such as over a period of weeks or months. Carriers may be configured for permanent implantation into a tumor bed, such as to provide radioactive energy to a treatment surface surrounding an area where a tumor has been removed in order to treat any remaining malignant tissue. Carriers can be composed of various materials and take on various shapes and sizes. Examples carriers, such as carriers having various sizes, shapes, configurations, etc., are included in the following patents and patent applications, each of which is hereby incorporated by reference in its entirety and for all purposes:
U.S. patent application Ser. No. 14/322,785, filed Jul. 2, 2014, now U.S. Pat. No. 8,876,684, entitled “Dosimetrically Customizable Brachytherapy Carriers and Methods Thereof In The Treatment Of Tumors,” and
U.S. patent application Ser. No. 14/216,723, filed Mar. 17, 2014, now U.S. Pat. No. 9,492,683, entitled “Dosimetrically Customizable Brachytherapy Carriers and Methods Thereof In The Treatment Of Tumors.”

Tile Carrier (also referred to as “Tile”): type of carrier that is substantially planar and generally maintains a two-dimensional planar geometry when placed in a tumor bed. Depending on the material of the tile, though, the tile may be malleable such that the tile can be deformed by bending in order to better conform to a tumor bed. For example, for tiles comprising collagen (and/or other malleable materials), the tiles may be substantially bent as placed in or on a treatment surface (and/or when pressed against the treatment surface) to conform with the shape of the treatment surface, such as a post-operative tumor bed.

Custom Carrier: a carrier having one or more non-planar surfaces, such as a spherical shape or having a spherical portion. Examples of custom carriers include Spherical Carriers, Gore Carriers, and Star Carriers, noted below, as well as other custom carriers discussed herein.

Spherical Carrier (or “GammaSphere”): a substantially radially symmetrical body around an axis. A spherical carrier may also include a non-spherical portion, such as a tapered portion that extends from a spherical portion. Examples of other variations of spherical carriers is discussed in Co-pending provisional application No. 63/163,583, filed Mar. 19, 2021 and entitled “Custom Brachytherapy Carriers,” which is incorporated by reference in its entirety and for all purposes.

Gore Carrier (also referred to as “Gore”): type of carrier that is 3-dimensional and conforms to the tumor bed while maintaining the geometry necessary for an effective implant. In some embodiments, gores are initially planar and are reconfigured to take on a 3-dimensional shape, such as to form a hemispherical surface that may be placed into a similarly shaped tumor cavity. Gore Carriers are further discussed in U.S. Pat. No. 8,876,684, entitled “Dosimetrically customizable brachytherapy carriers and methods thereof in the treatment of tumors,” filed on Jul. 2, 2014 as application Ser. No. 14/322,785, which is hereby incorporated by reference in its entirety and for all purposes.

Star Carrier (also referred to as “Star” or “arm-based carrier”): type of carrier that assumes a conformable 3-dimensional shape when arranged and placed into an operative cavity or similar space and conforms to the treatment environment while maintaining the geometry necessary for an effective implant. However, in some embodiments, Star carriers may be used in their initial planar state to cover a relatively flat tumor or tumor bed area. Star carriers are further discussed in U.S. Pat. No. 9,492,683, entitled “Dosimetrically customizable brachytherapy carriers and methods thereof in the treatment of tumors,” filed on Mar. 17, 2014 as application Ser. No. 14/216,723, which is hereby incorporated by reference in its entirety and for all purposes.

Loader: a device that aids in placement of radioactive seeds in carriers, such as via injection of seeds into carriers. A loader, also referred to herein as a “loading device,” may include multiple components, such as to hold a carrier in place and guide a delivery device (e.g., a needle or injector) into the carrier in order to place a seed at a precise location in the carrier. The “Loader Patents” refers to U.S. patent application Ser. No. 13/460,809, filed Apr. 30, 2012, now U.S. Pat. No. 8,939,881, entitled “Apparatus For Loading Dosimetrically Customizable Brachytherapy Carriers,” and U.S. patent application Ser. No. 14/696,293, filed April 24, 205, entitled “Apparatus and Method for Loading Radioactive Seeds Into Carriers,” which are each hereby incorporated by reference in their entirety for all purposes, describe several embodiments of loaders. As discussed further herein, loaders may be operated manually, such as by human operators, or may be fully automated, such that carriers can be loaded with seeds using an automated process. Alternatively, loaders may be configured to be automated in part and require manual operation in part.

High Z Materials: any element with an atomic number greater than 20, or an alloy containing such materials.

Shielding Material: any material that restricts movement of radioactive particles, such as by absorbing, reflecting, and/or scattering radioactive particles, such as with a high Z material. The term “shielding,” as used herein, generally refers to any mechanism of preventing radiation from moving through and exiting a corresponding shielding material, such as by the shielding material absorbing, reflecting, or otherwise blocking the radiation. Shielding materials in various forms may be used in the various embodiments discussed herein. For example, a shielding material may be in the form of a particle, wire, rod, cylinder, bar, sheet, liquid, solution, foam, or any other form in which a material having radiation absorbing and/or reflecting properties is possible.

Hot Carrier: a carrier that is loaded with a radioactive source.

Cold Carrier: a carrier that is not loaded with a radioactive source, such as a carrier prior to loading of a radioactive seed.

Proxy carrier: typically a cold carrier that can be placed as part of an implant planning process, such as to evaluate location, fit, and expected dosimetric distribution that would result if the cold carriers are replaced with hot carriers. For example, using the implant placement system described herein, as a user adds, modifies, removes, and/or moves physical proxy carriers around a treatment surface of a patient, a user interface depicting an expected dose to both a treatment area (where radiation is desired) and surrounding normal tissue areas (where radiation is not desired) is displayed. With the expected dose displayed and updated in close to real time (or real time), the surgeon or doctor is able to interactively adjust the types, strengths, configuration, and/or locations of proxy carriers until an optimate dosimetric distribution is achieved, and then replace the proxy carriers with hot carriers.

As discussed further herein, the implant placement system is configured to determine placement information (e.g., location and orientation of the proxy carrier with reference to a fixed origin point, or “reference position” on the treatment surface or other object) of each proxy carrier for use in calculating an expected dosimetric distribution. Proxy carriers range from “dumb” proxy carriers that are essentially cold carrier without any electronics or logic to “smart” proxy carriers that include sophisticated electronics that communicate placement information to the implant placement system, such as via wireless communications. Each type of proxy carrier may have advantages and disadvantages.

For example, a dumb proxy carrier may comprise only a collagen, or other bio-resorbable material, that is sized to match the outer dimensions of a hot carrier. Thus, the cost and the level of care required for use and maintenance of such a proxy carrier is very low. However, other proxy carrier detection components may be necessary to determine placement information of a dumb carrier. For example, the implant placement system may include and/or communicate with one or more sensors, such as cameras, lasers, probes, and/or other electronic devices, configured to provide sensor data that allows the implant placement system to determine placement information of each of the dumb carriers. Several examples of proxy carrier detection components are discussed herein.

In contrast to the more extensive proxy carrier detection components that may be needed to determine placement information of dumb proxy carriers, a smart proxy carrier may communicate its placement information (e.g., location and orientation with reference to a fixed origin point within the treatment area) directly to the implant placement system. For example, a smart proxy carrier may include one or more components such as accelerometers, gyroscopes, GPS receivers, microprocessors, wireless communication components, battery, and/or other components that are usable to determine and communicate placement information of the smart proxy carrier to the implant placement system.

Example implementations herein that do not specify proxy carrier capabilities (e.g., dumb or smart) may be implemented using a dumb, smart, or anything between dumb and smart, proxy carrier.

Carrier Characteristics: any attributes, properties, settings, or configurations of a carrier. Carrier characteristics may indicate a size, shape, and radiation dose. For example, carrier characteristics of a proxy carrier may indicate a size and shape of both the proxy carrier and a corresponding hot carrier that will replace the proxy carrier. Even though the proxy carrier does not include a radiation source, the carrier characteristics of a proxy carrier may include a radiation dose indicating the level of radiation of a radiation source of the hot carrier that will replace the proxy carrier. In this way, the expected dosimetric distribution from one or more hot carriers may be determined based on the carrier characteristics (e.g., radiation dose) of proxy carriers, before the hot carriers are implanted. Carrier characteristics may include additional characteristics, such as a carrier material, radioactive shielding of the carrier (which may not actually be present in the proxy carrier), location of a radiation source within the hot carrier (e.g., depth of the radiation source within a thickness of a tile carrier), a unique identifier, and/or any other characteristics of the proxy and/or hot carrier.

Dosimetry: a process of measurement and quantitative description of the radiation absorbed dose (e.g., rad) in a tissue or organ.

Calculated Dose: An amount of radiation or chemicals that will reach the tissues as a result of the placement of physical implants. For example, in the case of radiation implants, the (calculated) dose to tissue may be calculated using equations that account for the strength of each implant, its distance from the position for which the dose is calculated, and the intervening tissues, where the dose calculation may account for distance, radiation scatter, radiation absorption, and/or any other relevant factor. In the case of implants that release chemical agents, the calculated dose in different locations may be calculated based on factors such as the rate of release of the agent from the implant, tissue metabolism, solubility, diffusion, tissue perfusion, and/or any other relevant factor.

Dosimetric Plan: a description of how to achieve a particular dosimetric intent (e.g., a prescribed radiation dose and/or other radiation treatment objectives) for a particular patient, associated with a particular clinical condition, and/or for use in a particular surgical cavity, etc. Dosimetric plans may have varying levels of specificity. For example, a detailed dosimetric plan may be generated by complex dosimetry planning software and may indicate position, quantity, radioactive strength, etc., for placement of each of multiple radioactive carriers on a treatment surface of a patient. Another dosimetric plan may include only a dosimetric intent, such as only a total desired radiation dose. For example, dosimetric intent may not include specific brachytherapy parameters (e.g., a specific quantity and strength of radioactive implants), but rather may indicate only a total radiation dose to be delivered to a particular anatomical area of patient, and a dosimetric plan (e.g., placement and characteristics of radioactive carriers) may be determined in real-time intra-operatively. Regardless of the level of detail in a dosimetric plan, through placement of proxy (cold) carriers, a surgeon may safely experiment with quantity, placement, and other characteristics of hot carriers until the prescribed radiation dose is achieved, without exposing the patient (or others) to any radiation. As discussed further herein, this may be accomplished through calculation of a real-time expected dosimetric distribution based on the surgeon's placement of proxy carriers (e.g., cold carriers) in the actual locations and orientations as corresponding hot carriers may be placed. In this way, a dosimetric plan may be dynamically updated and/or entirely created intra-operatively using proxy carriers.

Deformation software: software that may be used to account for intra-operative cavity dynamics. For example, preoperatively acquired images (e.g., CT, MRI, or fusion data) may be mated with the optical based cavity dimensions, to generate deformation data indicating expected changes to the patient anatomy shown in the preoperative imaging as a result of the resection. For example, deformation software may calculate where a nerve might move when resection occurs, so that the dosimetric plan and/or placement planning may be developed to avoid the expected location of the nerve after resection (rather than the current location of the nerve prior to surgery). The deformation data may then be used by dosimetry software and/or the surgeon/doctor that is implanting the carriers, to provide an optimized dosimetric plan. Depending on the implementation (e.g., the type, size, location, etc. of the resection cavity, if any), deformation software may not be necessary.

Implant placement system (also referred to herein as a “Dose Feedback System”): One or more computing systems that aids a user and/or other system (e.g., a robotic surgical instrument) in placing implants in a surgical site, such as to implement a dosimetric plan. The system may execute placement software that generates visualizations of locations, types, strengths, and/or configurations of implants overlaid on live anatomical images of the patient. The planning software may further overlay an expected dose (e.g., in the form of isodose lines or curves) on images of the patient anatomy to show a current expected radiation dose distribution (e.g., if the proxy carriers are replaced with hot carriers). Thus, the placement system allows users (e.g., surgeons, doctors, nurses, etc.) to interactively view and make adjustments to proxy carriers, until a desired dosimetric distribution is achieved, all without additional radiographic or MRI re-imaging.

Planning image: An image or video of the anatomy, such as a treatment surface of a patient, that is viewable by a user, such as to generate a dosimetric plan and/or to indicate an expected dosimetric distribution associated with proxy carriers. A planning image may include one or more pre-operative and/or intra-operative images or videos that depicts the anatomy from various views or in 3D, for example a series of MRI or CT scans through the anatomy of interest. In some implementations of the implant placement system, a preoperative planning image is not necessary. In such embodiments, an intraoperative live planning image of the patient's anatomy may be overlaid with an expected dosimetric distribution that allows the user to generate and/or refine a dosimetric plan.

Isodose curve (also referred to herein as an “isodose line”): a graphical indication, such as a line, indicating points of equal dose about a radiation source, such as a radioactive seed. Multiple isodose curves may illustrate an expected dosimetric distribution around a radiation source at regular intervals of absorbed dose, or other intervals. In some implementations, isodose curves indicate percentages of a dose that are absorbed along the isodose curve. Isodose curves may be calculated for various tissue depths, such as at the treatment surface and/or at multiple tissue depths.

Expected Dosimetric Distribution (also referred to herein as “Expected Dose” or “Estimated Dose”): Calculated radiation dose(s) across an area of tissues that are expected to result from replacement of one or more proxy carriers on a treatment area of a patient with hot carriers having characteristics (e.g., dose, shielding, size, etc.) associated with the corresponding proxy carrier. The expected dose can be visually represented by a group of isodose curves each associated with different calculated dose ranges. An expected dose visualization may include various patterns and/or fills between adjacent isodose curves, such as varying colors, shading, patterns, and/or other visual indicators that represent corresponding dose ranges. In some implementations, dosage ranges between respective isodose curves may be represented by varying densities of dots, hatching, and/or other patterns. In some implementations, different colors provide a more distinct separation between isodose curves. For purposes of illustration, certain of the figures use varying pattern densities to indicate varying calculated dose ranges, but any other representation of varying dose ranges, such as different colors, may be used. In other implementations, information about an expected dosimetric distribution may be provided via various technologies, such as any 2D images, audio feedback, 2D or 3D imagery via an augmented or virtual reality headset, xray goggles, surgical scope filters, or any other interfaces.

Placement Information: information about a carrier (e.g., a proxy carrier or a hot carrier) indicating position of the carrier. Placement information may include, for example, a three-dimensional location and orientation of a carrier with reference to a fixed origin point in a treatment room (e.g., surgical operating room or, more generally, an implant placement room). For example, placement information may include an X, Y, and Z distance from a specific portion of a treatment surface, such as an upper boundary of a resection cavity. Placement information may include 3D coordinates for one or more points (e.g., three points) on the carrier so that orientation, as well as position, of the carrier is defined also. For example, placement information of a rectangular carrier (e.g., a tile) may include three sets of three-dimensional coordinates (e.g., distances from an origin point) for each of three corners of the rectangular carrier (e.g., [X_{corner1}, Y_{corner1}, Z_{corner1}], [X_{corner2}, Y_{corner2}, Z_{corner2}], [X_{corner3}, Y_{corner3}, Z_{corner3}]). In some embodiments, placement information may include fewer three-dimensional coordinates, such as in the case of a spherical carrier that has a substantially uniform shape at any orientation. As discussed further herein, placement information may be determined based on analysis of sensor data, such as an image or a video stream (e.g., a series of images) of the implantation site, location data from a probe touching a reference marker on a carrier (e.g., reference markers on corners or other locations of the carrier), location data received from a smart proxy carrier, and/or any other sensor data.

### Example System Implementations and Embodiments

Described herein are example implementations, embodiments, and/or components of a real-time or near real-time intra-operative radiation dose feedback system (also referred to herein as an implant planning system). The dose feedback system (or any components thereof) can be implemented to improve and/or ensure that a proper amount of radiation is delivered to a patient by, for example, carriers (e.g., tile carriers). The dose feedback system can be implemented during medical surgeries, operations, procedures, etc. In one example, the dose feedback system may be implemented during a medical procedure to remove a tumor (e.g., cancerous growth) from the body of a patient before the cavity of the tumor bed has been closed. Advantageously, the dose feedback system may allow a medical professional (e.g., surgeon) to more accurately achieve a prescribed radiation of a dosimetric intent or a more detailed dosimetric plan through adjustments to position, dose, quantity, etc. of carriers prior to terminating the medical procedure (e.g., cavity of tumor bed is closed and/or patient is brought out from under anesthesia) at which point adjusting the radiation dose may be difficult or impossible. Thus, because the dose feedback system may indicate, during a medical procedure, an expected dosimetric distribution to a medical professional, the medical professional may not need to use radiographic and/or MRI re-imaging to verify a delivered radiation dose after termination of the tumor removal surgery and/or carrier placement procedure.

The dose feedback system can provide feedback to a medical professional of an expected dosimetric distribution that is calculated based, at least in part, on an expected placement of carriers in a patient (e.g., in a tumor bed). Based on the expected dosimetric distribution, the medical professional may adjust the carrier placement (e.g., to change the pre-operative dosimetric plan) and update the expected dosimetric distribution until a dosimetric intent and/or dosimetric plan is achieved.

In some embodiments, the dose feedback system can be implemented prior to commencement of a medical procedure, for example to allow a medial professional to prepare for the procedure by preparing, planning, and/or optimizing a delivered radiation dose. For example, a user may use the dose feedback system to generate a dosimetric plan using proxy carriers that are placed on a pre-operative anatomy of the patient and/or on a non-patient analog (e.g., a plaster skull with a cavity that approximates the expected resection cavity of the patient).

The dose feedback system may be implemented by medical professionals such as doctors, physicians, surgeons, etc. and may be implemented to provide medical care to patients. The dose feedback system can also be implemented by teachers, educators, students to provide training, education, or practice. For example, medical students may use the dose feedback system to practice optimizing a delivered radiation dosage without performing an operation on an actual patient.

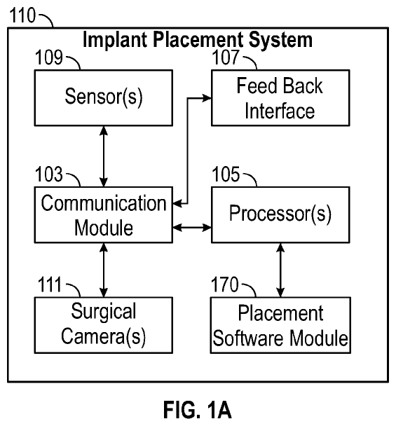

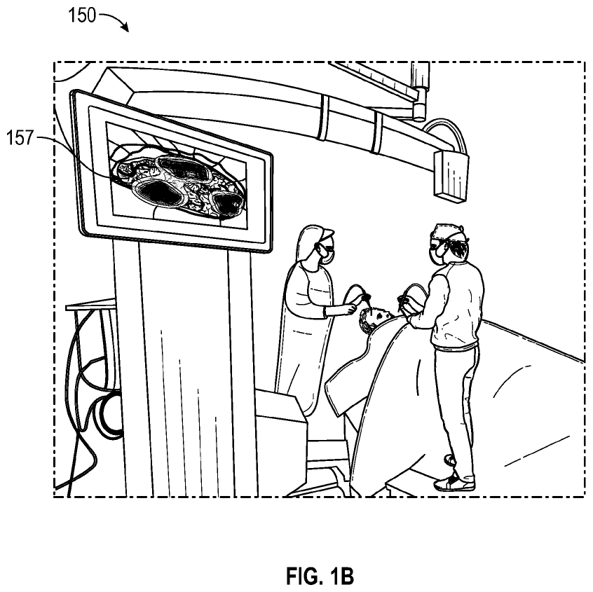

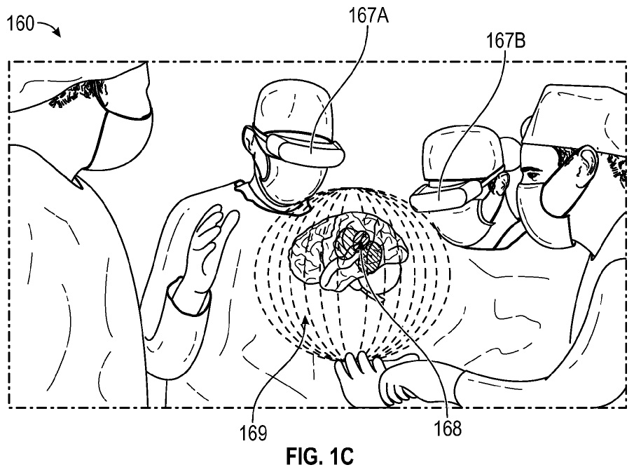

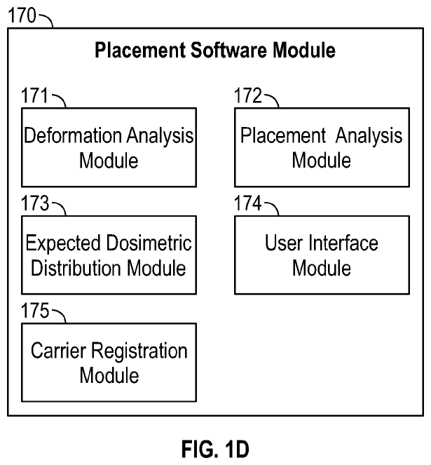

FIG. 1A is a schematic diagram illustrating an example real-time or near real-time intra-operative radiation implant placement system 110. In the example of FIG. 1A, the implant placement system 110 includes one or more computer processors 105, sensors 109, a communication module 103, a feedback device 107, one or more surgical cameras 111, and a placement software module 170. Depending on the embodiment, the implant placement system 110 may include fewer or additional components. For example, in some embodiments, the implant placement system may not include a surgical camera.

The implant placement system 110 includes a communication module 103. The communication module 103 can facilitate communication (via wired and/or wireless connection) between other components of the implant placement system 110 and/or communication with separate devices, systems, and/or networks. For example, the communication module 103 can be in communication with the sensor(s) 109, one or more smart proxy carriers, surgical camera(s) 111, feedback device 107, and/or processor(s) 105 over any of a variety of communication protocols. The communication module 103 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 103 can allow data and/or instructions to be transmitted and/or received to and/or from the other components of the implant placement system 110. The communication module 103 can comprise a wireless transceiver, an antenna, a near field communication (NFC) component, or the like.

The implant placement system 110 includes one or more processors 105. The processor(s) 105 can include hardware processors and can be configured, among other things, to process data, execute instructions to perform one or more functions, and/or control the operation of the implant placement system 110. For example, the processor(s) 105 can execute the placement software module, which may include software that causes the system to perform various operation (e.g., see FIG. 1D). The processor(s) 105 may process data received from the communication module 103 and can execute instructions to perform functions related to analyzing and/or transmitting such data. The processor(s) 105 can be configured to receive data or information from a system, device, or network external to the implant placement system 110 such as via the communication module 103.

The example implant placement system 110 includes a placement software module 170 comprising executable software instructions. The placement software module 170 can include various modules, that when executed, perform various functions or operations, such as described with reference to FIG. 1D. The placement software module 170 can be executed by any computing device or system configured to execute software instructions, such as the one or more processors 105, a computing device or system that is remote to the implant placement system and/or the implant placement system 110, the cloud, and the like.

The example implant placement system 110 includes a feedback device 107. The feedback device 107 may include systems or devices configured to output auditory and/or visual indicators. For example, the feedback device 107 may include speakers for outputting one or more auditory signals and/or may include one or more displays or screens for rendering or displaying one or more images or videos. In some embodiments, the feedback device 107 include a screen such as a computer monitor, a television monitor, a laptop screen, a handheld device, a mobile device such as a smartphone, or the like. In some embodiments, the feedback device 107 may include a virtual reality (VR) or augmented reality (AR) system or devices, such as a VR or AR headset. In some embodiments, the feedback device 107 may be configured to render a 3 dimensional image on a display. In some embodiments, the feedback device 107 may be interactive. For example, the feedback device 107 may include a touch screen configured to receive user input via user touch on the feedback device 107. In some embodiments, the feedback device 107 may be remote to other devices or components of the implant placement system 110, such as a few feet away or in another part of the world. In some embodiments, the feedback device 107 can include a projection device (e.g., a high definition video projector in an operating room) configured to transmit a dosimetric plan and/or expected dosimetric distribution onto a treatment surface of the patient.

The feedback device 107 may be in communication with the communication module 103. The feedback device 107 may receive, via the communication module 103, information from the processor(s) 105. The feedback device 107 may output one or more visual or auditory indicators according to data and/or instructions received from the processor(s) 105. For example, the feedback device 107 may output an image or video of an operation site (which may be adjusted to account for deformation resulting from the operation) in combination with information (e.g., images) relating to a dosimetric intent, an expected dosimetric distribution, or proxy carrier placement. In some embodiments, the feedback device 107 may output one or more visual or auditory indicators when a proxy carrier has been properly placed according to a dosimetric plan and/or to achieve a desired radiation dosage (e.g., in satisfaction of a dosimetric intent). In some embodiments, the feedback device 107 may output one or more visual or auditory indicators when a carrier (e.g., a proxy carrier or a hot carrier) has been placed with a position and/or orientation that would violate a desired radiation dosage or dosimetric intent.

The implant placement system 110 may include one or more sensors 109, such as proxy carrier detection sensors. The sensor(s) 109 may include lasers, cameras (e.g., high speed cameras, 3D depth cameras), probes or the like. The sensor(s) 109 can be configured to obtain information relating to a placement of a proxy carrier. For example, the sensor(s) 109 can be configured to obtain information relating to a position, location, and/or orientation of proxy carriers in a surgical site of a patient. The sensor(s) 109 can communicate proxy carrier placement information to the processor(s) 105 via the communication module 103. In embodiments where smart sensors are used, the implant placement system 110 may not include sensors 109, and the placement information of the smart sensors may be transmitted directly to the communication module 103.

The example implant placement system 110 includes one or more surgical cameras 111. The surgical camera(s) 111 can include any camera or video capture device used during a surgery or medical procedure, such as a downward facing camera mounted on the ceiling or on a moveable frame in an operation room, or smaller camera, such as an endoscope, that is closer to the surgical site. The surgical camera(s) 111 can capture image and/or video data of an operation site (e.g., a tumor bed) and provide the data to the implant placement system 110 via the communication module 103. The implant placement system 110 may process the data (e.g., by the processor 105) and output an image and/or video of the operation site via the feedback device 107. The image and/or video of the operation site may be modified (e.g., by the processor 105), as discussed herein, to include additional information such as a dosimetric intent, an expected dosimetric distribution, an expected carrier placement, and/or deformation adjustments.

The implant placement system 110 may determine an expected dosimetric distribution that indicates estimated radiation doses expected from placement of hot carriers at the determined locations of each of one or more proxy carriers. The expected dosimetric distribution may be based, at least in part, on the placement (e.g., position, location, orientation etc.) of the proxy carrier(s) in a patient, which placement may be determined by the senor(s) 109 or sensors within a smart carrier. The implant placement system 110 may output the expected dosimetric distribution via the feedback device 107. In some embodiments, radiation doses of each carrier used in a particular surgical procedure are the same and may be provided by a user of the system or pre-set in the implant placement system 110 software. Radiation dose of a carrier may be determined as based on a quantity of radioactive seeds (e.g., CS-131 seeds) that are embedded within a carrier. Thus, calculation of the expected dosimetric distribution may use a same radiation dose for each of the carriers in such an embodiment. In some examples, carriers may have different radiation dosages. For example, a first carrier may have four embedded radioactive seeds, while a second carrier may have only a single embedded radioactive seed. In some implementations, the implant placement system 110 determines radiation dose of each carrier based on a detected characteristic of the carrier, such as a marking, size, shape, texture, etc., that may be detected by one or more sensors of the system. For example, a camera may detect markings on a carrier (e.g., dots on a surface of the carrier indicating the quantity of embedded radiation sources, such that four dots would indicated four implanted radiation sources) and customize the dosimetric distribution based on the determined radiation doses of the individual carriers.

FIGS. 1B-1C illustrates example embodiments of dose feedback systems implemented during a medical procedure. FIGS. 1B-1C are provided as examples and are not intended to be limiting. In some embodiments, one or more features of FIGS. 1B-1C may be combined. For example, a dose feedback system may be implemented with one or more feedback devices of various types.

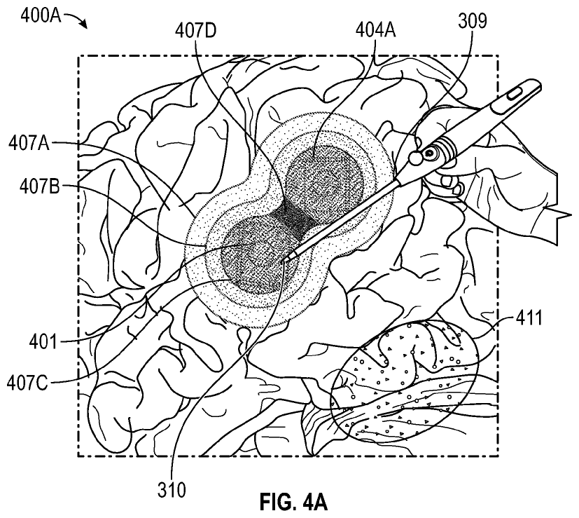

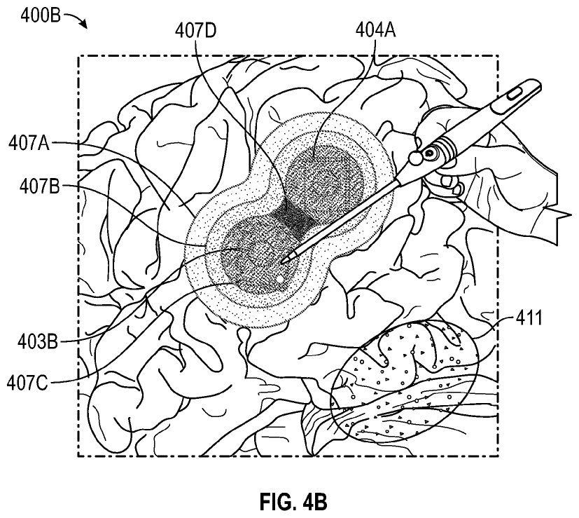

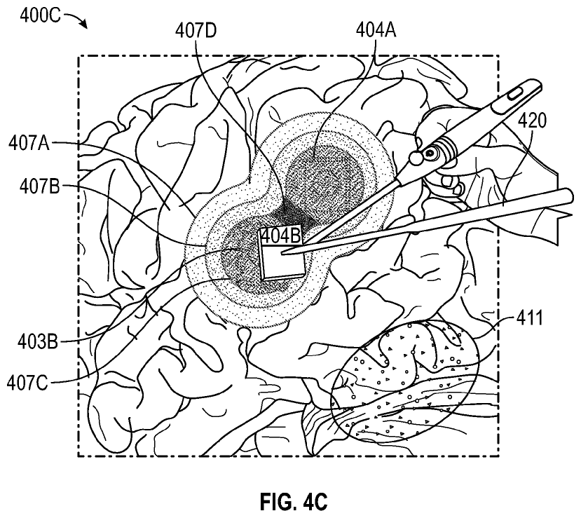

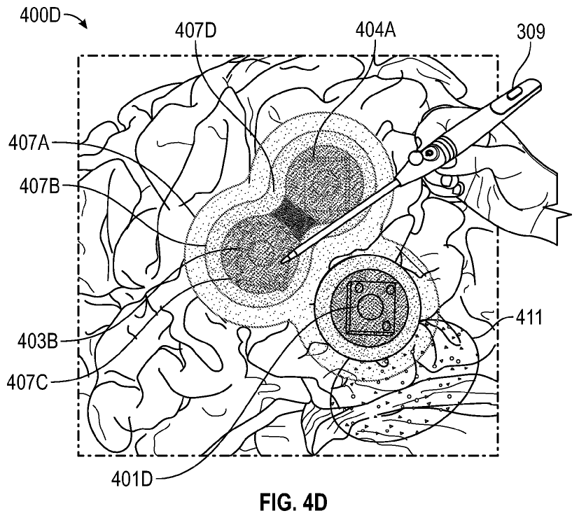

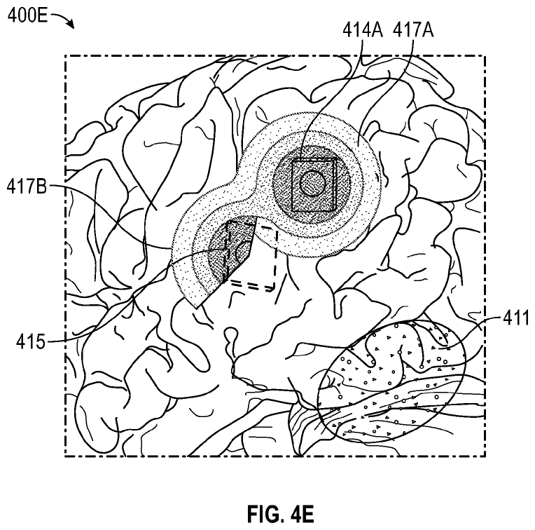

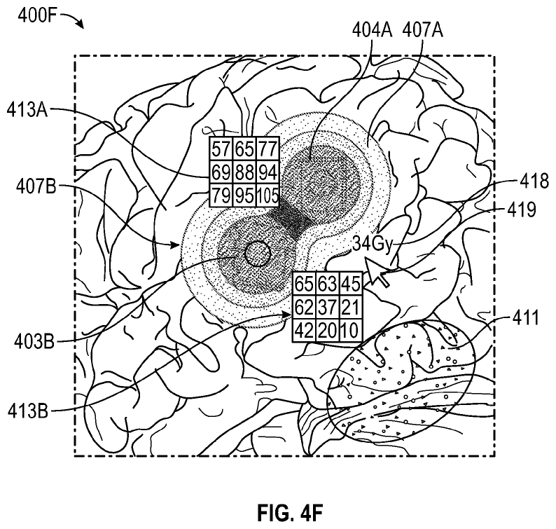

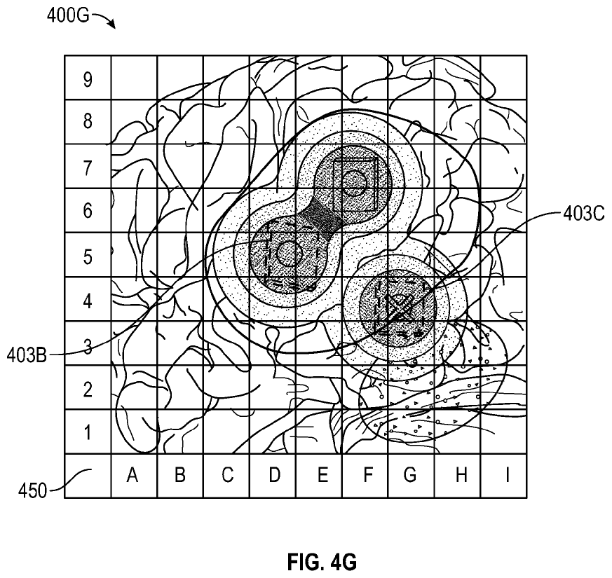

FIG. 1B illustrates an example operating room 150 implementing a dose feedback system during a medical procedure. As shown, a feedback device 157, which includes a display screen, displays a visual representation of the operation site (e.g., the tumor bed). In different implementations, the feedback device may include various display devices, such as one or more of a computer display, tablet or mobile device, television, AR or MR headset, a projector, etc. The visual representation may include real-time images or video of the surgical site, as captured by, and received from, a surgical camera such as a downward facing camera or an endoscope. In the example of FIG. 1B, the visual representation displayed via the feedback device 157 also includes an expected dosimetric distribution associated with one or more proxy carriers located in the treatment area. The information provided via the feedback device 157 may facilitate optimizing a delivered radiation dosage by dynamically providing the expected dosimetric distribution to medical professionals as proxy carriers are added and/or repositioned within the treatment area. In some embodiments, the feedback device 157 can be configured to display any of the example planning images 400A-400H, or features thereof, individually or in combination, as shown and discussed with reference to FIGS. 4A-4G.

As shown in the example embodiment of FIG. 1B, the feedback device 157 may include a large screen that can be viewed by one or more persons in a room simultaneously. In some embodiments, the feedback device 157 may include multiple screens displaying the same thing such as to allow multiple people (who may be remote to one another) to individually view the same thing and/or to allow multiple people in a same room to view the same thing. As noted elsewhere, the feedback device may include other devices, such as an AR headset, projected image, etc.

FIG. 1C illustrates an example operating room 160 implementing another embodiment of a dose feedback system during a medical procedure. As shown, the feedback device 167 (e.g., feedback devices 167A, 167B) include devices configured to display a virtual or augmented reality, such as a VR/AR headset, goggles, or glasses. For example, a virtual reality device may display images or videos to a user without also displaying and/or permitting the user to view the physical world. Alternatively, an augmented reality device may display an virtual content overlaid on the physical world. For example, an augmented reality feedback device 167 may display images or videos superimposed on a view of the physical world, and appear, to the perspective of the user, to be part of the physical world.

In the example of FIG. 1C, a first medical professional wears a first feedback device 167A and a second medical professional wears a second feedback device 167B. The feedback devices 167A and 167B may display the same or similar videos or images, from the perspective of the respective wearers, so that the two medical professionals view the same or similar thing.

The example of FIG. 1C shows a virtual display 169 that may not exist in the physical world, but may only be viewed with the feedback devices 167A, 167B. In this example, the virtual display 169 includes an image of a surgical site (e.g., before, during, and/or after a medical procedure). In some embodiments, virtual display 169 may include adjustments to account for deformation to the tissue of the surgical site that have resulted and/or are expected to result from the surgical operation. In some embodiments, the virtual display 169 may be a 3 dimensional representation of the surgical site. In this example, the virtual display 169 also includes an expected dosimetric distribution 168, such as based on one or more proxy carriers that are positioned in the surgical cavity (intra-operative) and/or are part of a dosimetric plan (pre-operative). In this example, the expected dosimetric distribution 168 is 3 dimensional. The virtual display 169 can include (e.g., display) other various elements, such as anticipated or actual carrier placements in the surgical site, sensitive tissue areas, and the like. In some embodiments, the feedback device 167 can be configured to display any of the example planning images 400A-400H, or features thereof, individually or in combination, as shown and discussed with reference to FIGS. 4A-4G, in two dimensions or in 3 dimensions.

A user may be able to interact with the virtual display 169. For example, a user's actions in the physical world, such as pointing to a space in the physical world corresponding to a virtual object included in the virtual display 169, may cause the virtual display 169 to update based on the user's actions. A user may be able to interact with and/or view the virtual display 169 prior to a medical procedure and/or during the medical procedure (e.g., intra-operatively in real-time) to facilitate placing carriers during the medical procedure. A user may be able to interact with and/or view the virtual display 169 after a medical procedure to review carrier placement and a resulting radiation dosage. In another embodiment, the 3D image and expected dosimetric distribution is a hologram that is viewable from any angle and without the feedback devices 167.

FIG. 1D is a block diagram illustrating example components of a placement software module 170 (e.g., FIG. 1A). The placement software module 170 can include executable software instructions or modules, that when executed, perform various functions or operations, as described herein. In this example, the placement software module 170 includes a deformation analysis module 171, a placement analysis module 172, an expected dosimetric distribution module 173, a user interface module 174, and a carrier registration module 175. In other embodiments, the placement software module may include fewer or additional software modules and/or the modules may be combined or further separated into additional function software modules.

The deformation analysis module 171 can receive images captured prior to commencement of an operation (e.g., pre-op images) which may include MRI images, X-ray images, camera images, or a combination thereof. The deformation analysis module 171 may process the pre-op images (e.g., according to deformation algorithms) to generate data relating to a real-time or present condition of the operation site. For example, tissue surrounding the operation site may shift during the operation (e.g., when the tumor is removed) and the deformation analysis module 171 may adjust the pre-op image to indicate expected deformations to tissue from the operation. The deformation adjusted pre-operative images may then be used in development of a dosimetric plan, such as to indicate sensitive tissue areas that are expected to move and/or change shape as a result of the surgery so that radiation to those expected sensitive tissue areas may be minimized. In some embodiments, the deformation analysis module 171 can determine a location of sensitive tissue in a surgical site such as a tumor bed before and/or after surgery has commenced. Sensitive tissues can include any tissue that is to receive no radiation, or a minimal amount of radiation, such as nerve tissue, brain tissue, and the like.

The placement analysis module 172 may be configured to determine placement information of proxy carriers, such as a location and orientation of the proxy carrier with reference to a fixed origin point (also referred to as a “reference position”) on the treatment surface (or position on patient bed, equipment in the room, walls, ceiling, floor, or any other physical object). The placement analysis module 172 may determine the placement information based, at least in part, on information received from the proxy carrier and/or information received from one or more sensors, such as lasers, cameras, probes, and the like.

The expected dosimetric distribution module 173 is configured to generate an expected dosimetric distribution of one or more hot carriers corresponding to respective proxy carriers positioned in or on the treatment area. The expected dosimetric distribution module 173 may determine the expected dosimetric distribution based, at least in part, on the determined placement information of the proxy carriers, such as may be determined based on sensor data (e.g., from sensors 109 or camera 111) and/or may be received from a smart proxy carrier. The carrier specifications (e.g., radiation strength, direction of expected emitted radiation, carrier size, etc.), may be used by the expected dosimetric distribution module 173 to develop a real-time estimate of dosimetric distribution associated with one or more carriers. In some embodiments, the carrier specifications may be determined by a carrier registration module 175 (discussed below).

In some embodiments, the expected dosimetric distribution module 173 can be configured to determine whether an expected dosimetric distribution is acceptable (e.g., sufficiently matches a dosimetric plan and/or meets a dosimetric intent). The expected dosimetric distribution module 173 can compare the expected radiation dosage to a prescribed radiation dosage, e.g., for each of multiple locations around a resection cavity, to determine an amount of correlation. If a difference between the expected and desired radiation doses is within a certain threshold (e.g., the amount of correlation of the expected radiation dosage and the prescribed radiation dosage is very high), the expected dosimetric distribution may be acceptable. Depending on the embodiment, the degree of matching of expected radiation dosage with a prescribed radiation dosage may be determined by the system (e.g., a default variance of plus or minus five percent may be allowed), may be provided by a prescribing radiation oncologist, and/or may be developed by one or more medical professionals involved with the tumor removal and/or carrier placement process. In some embodiments, if a prescribed radiation dosage to a particular area is outside of an acceptable range, the system may provide various alerts to the user and/or prevent the user from indicating the implantation process is complete.

In some embodiments, the expected dosimetric distribution module 173 can be configured to determine whether an expected dosimetric distribution is undesirable. For example, the expected dosimetric distribution module 173 may determine whether the expected dosimetric distribution violates a desired or intended radiation dose, such as when too much radiation is delivered to a normal tissue site. The expected dosimetric distribution module 173 may compare expected radiation doses with desired radiation doses for various portions of a tissue site. If a difference between the expected and desired radiation doses exceeds a certain threshold, the expected dosimetric distribution may be undesirable.

The planning image module 174 can be configured to receive, analyze, process, and/or transmit image data. For example, the planning image module 174 can receive data relating to images captured by a surgical camera such as an endoscope. The planning image module 174 can process the data from the surgical cameras and generate data to render a visual display of the operation site. As another example, the planning image module 174 can receive data relating to a dosimetric intent such as from a system, device, memory, database, manual user input, or remote network. The expected dosimetric distribution module 173 may process the dosimetric intent data to generate data to render a visual display of the dosimetric intent.

The planning image module 174 can process any combination of the information described herein. For example, the planning image module 174 can combine, merge, overlay, superimpose, or otherwise generate data for simultaneously displaying dosimetric intent data or images, expected dosimetric distribution data or images, proxy carrier or carrier placement data or images, pre-op data or images, deformation data or images, and/or operation site data or images.

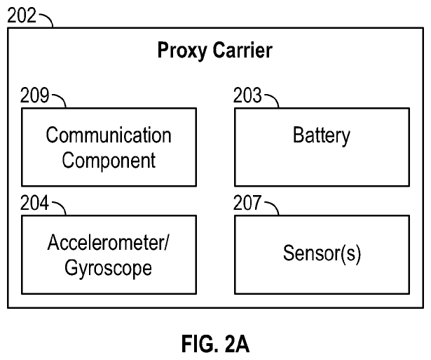

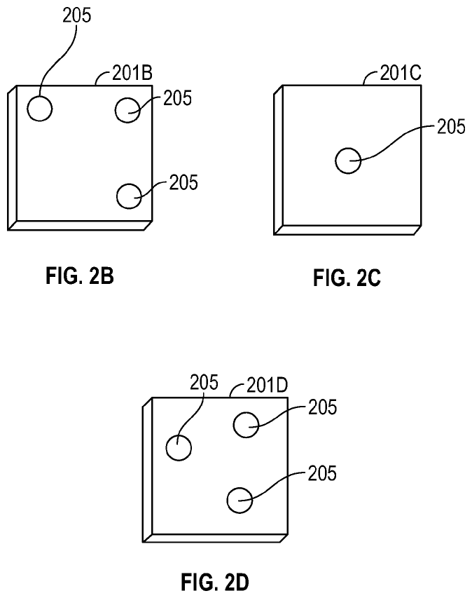

The carrier registration module 175 can receive, process, and/or analyze information relating to a hot carrier or proxy carrier. As an example, the carrier registration module 175 may receive and/or access information such as a magnitude of radiation emitted from a carrier, a carrier size, a direction of radiation emitted from a carrier such as may be affected by radiation shielding, a carrier identification, information relating to identification structures on the carrier (e.g., as shown and discussed with reference to FIGS. 2B-2D), and the like. The proxy carrier may be selected and/or configured with such information to match related characteristics of a carrier that is to be used during a medical procedure (e.g., placed in a tumor bed). In some embodiments, the carrier registration module 175 can receive such information directly from a proxy carrier, such as in embodiments where the proxy carrier is configured to communicate information. In some embodiments, the carrier registration module 175 may receive such information from a local or remote data store. The carrier registration module 175 may receive such information during a registration procedure or protocol.

### Example Carrier Embodiments and Implementations

In some embodiments, proxy carrier may include structural features used in conjunction with the implant placement system 110 to facilitate placement information of the proxy carrier. For example, a proxy carrier may include one or more of fiducial markers, fluorescent coatings, reflective coatings, electrical properties or characteristics, or mechanical properties and/or characteristics. Any of the foregoing, or combinations thereof, may be used by the implant placement system 110 to determine placement information of the proxy carrier. For example, a sensor 109 of the implant placement system 110, such as a camera, may capture images of a proxy carrier. The processor(s) 105 may process the images to identify the proxy carrier (e.g., by identifying fiducial markers, reflective coatings, unique electrical/mechanical properties etc. of the proxy carrier) and to determine placement information, such as a placement position and/or orientation of the proxy carrier, for example, in a tumor bed.

FIG. 2A is a block diagram illustrating an example embodiment of a proxy carrier 202. In this example embodiment, the proxy carrier 202 is a smart carrier that includes components configured to detect and communicate placement information of the carrier to an implant placement system. In this example, the proxy carrier 202 includes a communication component 209, a battery 203, an accelerometer and/or gyroscope 204, and/or one or more other sensors 207. The battery 203 may provide power to the other components of the proxy carrier 202. The accelerometer/gyroscope 204 may obtain information relating to a position and/or orientation of the proxy carrier 202. The sensor(s) 207 may include any type of other sensor, such as a pH meter or the like which may be configured to determine a pH level of surrounding tissue which may provide information relating to an infection of the surrounding tissue.

The communication component 209 may facilitate communication between the proxy carrier 202 and other devices or systems such as the implant placement system 110 described with reference to FIG. 1A. For example, the communication component 209 may be configured to communicate placement information to an implant placement system relating to the proxy carrier's 202, including, e.g., a position and orientation with reference to a defined point of the patient and/or the operating room. The communication component 209 can be configured to use any of a variety of wireless communication protocols, such as Wi-Fi (802.11x), Bluetooth®, Low Energy Bluetooth (BLE), ZigBee®, Z-wave®, cellular telephony, infrared, near-field communications (NFC), RFID, satellite transmission, proprietary protocols, combinations of the same, and the like. The communication component 209 can be embodied in one or more components that are in communication with each other. The communication component 209 can comprise a wireless transceiver, an antenna, a near field communication (NFC) component, or the like.

FIG. 2A is provided as an example as is not meant to be limiting. In some embodiments, the proxy carrier 202 may include more or less components than what is shown in FIG. 2A. For example, in some embodiments, the proxy carrier 202 may not include one or more of the components shown in FIG. 2A.

FIGS. 2B-2D are perspective views illustrating example embodiments of a proxy carrier 201 (e.g., proxy carrier 201B, 2010, 201D). The example proxy carriers 201 may include one or more markings 205 that serve as registration points that are usable to determine placement information of the proxy carrier. In the example of FIGS. 2B-2D, the proxy carriers 201B-201D are dumb proxy carriers, e.g., collagen with the indicated markings and no internal components. In other embodiments, markings 205 may be used in conjunction with smart proxy carriers in a similar manner to aid in determination of placement information of the smart proxy carriers. In some embodiments, hot carriers may include markings 205 configured to aid in registration of the hot carriers, which may remove the need for use of proxy carriers.

The markings 205 serve as registration points for the proxy carriers. The markings 205 may be prominent or readily visible, to facilitate detection of the markings 205 by a human users and/or by an implant placement system. The markings 205 may include a structural formation, such as an indent, a raised surface, a textured surface, a material that is a different material than the surface of the proxy carrier 201, a visible marking, and/or the like.

An implant placement system may be configured to detect the markings 205 of proxy carriers 201 and thereby determine placement information (e.g., location, position, orientation, etc) of the proxy carrier. For example, sensors, such as lasers, cameras, probes, etc. of an implant placement system may detect the markings 205. In some embodiments, the implant placement system may only receive information relating to the markings 205 and not information relating to the entire proxy carrier 201. Advantageously, this may simplify and/or reduce processing requirements for determining placement information for a proxy carrier 201. The implant placement system may be configured to extrapolate information relating to the entirety of the proxy carrier 201, from the marking 205 location information, based on a calibration of the implant placement system. For example, the implant placement system may be calibrated to process information relating to the marking 205 based on the assumption that the structure 205 is located at a geographically central location of a side surface of the proxy carrier 201. The implant placement system may be calibrated in any manner as required or desired depending on the marking 205 and their arrangement or location on the proxy carrier 201.

As shown in FIG. 2B, the example proxy carrier 201B includes three markings 205 positioned at 90-degree angles from one another on a surface of the proxy carrier 201B. Advantageously, the three markings 205 may provide information relating to the proxy carrier's 201B position, location and/or orientation which may be inferred or extrapolated from the positions, sizes, and/or other characteristics of the markings 205.

As shown in FIG. 2C, the example proxy carrier 201C includes one marking 205 at a geographically central location on a surface of the proxy carrier 201C. Advantageously, the geographically central marking 205 may provide information relating to the proxy carrier's 201C position or location, because the shape and/or position of the proxy carrier 201C may be known with respect to the position of the marking 205. Additionally, including only a single marking 205 may simplify and/or reduce processing requirements for determining proxy carrier 201 location based on marking 205 location because sensor(s) may only need to detect and/or processors may only need to determine the location information of the single marking 205, and/or because a human may only need to touch a probe to the single marking 205.

As shown in FIG. 2D, the example proxy carrier 201D includes three markings 205 on a surface of the proxy carrier 201D. None of the markings 205 are located at a geographically central location of the surface of the proxy carrier 201D. Advantageously, the three markings 205 may provide information relating to the proxy carrier's 201D position, location and/or orientation which may be inferred or extrapolated from the positions of the markings 205.

In some embodiments, a proxy carrier 201 may include any number of markings 205, such as two markings 205, or more than three markings 205. In some embodiments, the marking 205 may be located on more than one surface of the proxy carrier 201. In some embodiments, the marking(s) 205 may be arranged and/or located on a surface of the proxy carrier 201 according to any combination of the examples provided herein.

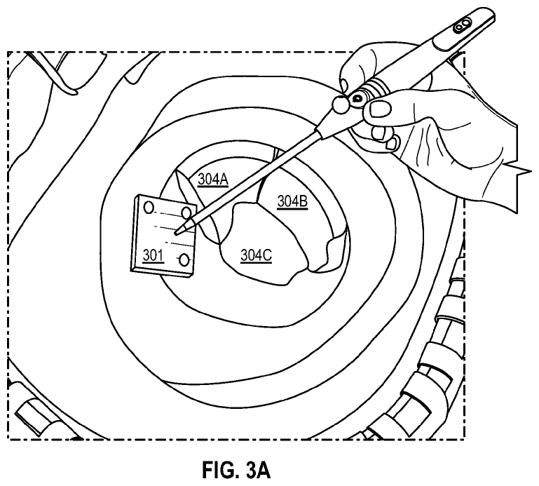

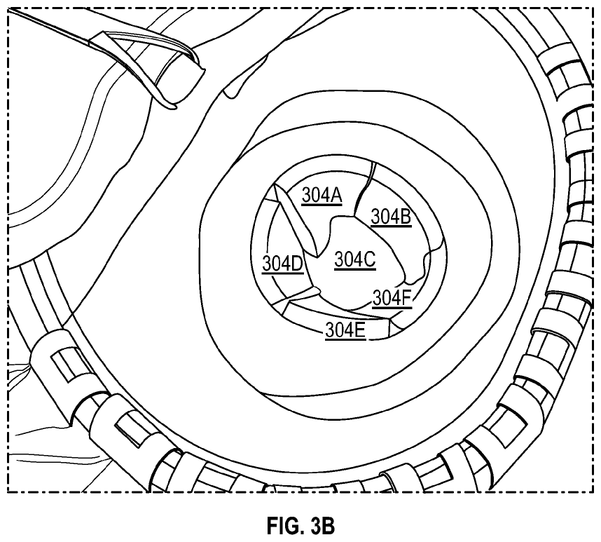

In some embodiments, the markings 205 may include an indentation and/or be configured to receive the tip of a probe, for example, as shown in FIG. 3A. The probe may be included as part of an implant placement system and may include sensors or processors for determining and transmitting information relating to the location of the probe (e.g., the tip of the probe). When the tip of the probe is in contact with a marking 205, the probe may determine and communicate information relating to the location of the tip of the probe to a communication module to be further processed and analyzed by the implant placement system. In some embodiments, the probe may determine and communicate said information continuously or periodically. In some embodiments, the probe may determine and communicate said information in response to a user command. For example, a user may press a button (e.g., on the probe) when the user is satisfied that the tip of the probe is properly positioned with reference to a marking (e.g., touching a marking), which may cause the probe to determine the location of the tip at that time and to transmit that information. In some embodiments, the probe may determine and communicate said location information automatically. For example, in some embodiments, the marking 205 may be made of different material than the rest of the proxy carrier 201, such as an electrically conductive material. The probe may be configured to detect when the tip of the probe contacts the material of the marking 205 and, in response, determine and/or transmit location information.

In some embodiments, markings 205 may include a material that is easily identifiable by a camera (or other sensor) such as a reflective material which may facilitate detection of the marking 205 by an implant placement system.

In some embodiments, a proxy carrier may be made of bio-compatible and/or bio-resorbable materials such as collagen. In some embodiments, a proxy carrier may not be made of bio-compatible materials because the proxy carrier may not permanently remain in a patient's body. For example, a smart proxy carrier may include sensors and/or other electronic components (that are not bio-resorbable) that allow the smart proxy carrier to determine its placement information and transmit such placement information to the implant placement system.

In some embodiments, the proxy carriers 201 discussed with reference to FIGS. 2B-2D may be either dumb or smart carriers. For example, a smart proxy carrier may include markings such as those in FIGS. 2B-2D and some or all of the components of example smart proxy carrier 202 (FIG. 2A). Additionally, in some implementations the features and components of the example proxy carriers 201 may be included in hot carriers, such as to confirm location and/or placement of hot carriers on a treatment service.

FIG. 3A illustrate example implementations of a proxy carrier 301 in a tumor bed of a patient. A medical professional may have placed carriers 304 (e.g., carriers 304A, 304B, 304C) in a tumor bed of a patient as shown. In this example, the carriers 304 are hot carriers, each including one or more radiation sources. In other embodiments, the carriers 304 may be cold proxy carriers, not including radiation sources. Prior to placing another hot carrier, the medical professional may place the proxy carrier 301 to facilitate determination of the optimum placement. The proxy carrier 301 may be a same or similar shape, size, and/or form as the carriers 304. The proxy carrier 301 may include any of the structural and/or functional features described herein, for example, with reference to FIGS. 2A-2D.

After the medical professional has placed the proxy carrier 301 in the tumor bed, the medical professional may perform a carrier registration process to determine placement information (e.g., location, orientation) of the proxy carrier 301, as well as update an expected dosimetric distribution with the placement information and radiation delivery characteristics of the corresponding hot carrier. For example, the medical professional may touch a tip 310 of the probe 309 to one or more of the markings on a surface of the proxy carrier 301. The probe 309 may include sensors to determine a location of the tip of the probe 309. The probe 309 may be configured to transmit data (e.g., tip location) to a remote system or device of an implant placement system. The probe 309 may be configured to determine and/or transmit tip location information automatically or in response to an input. For example, the probe 309 may be configured to determine and/or transmit tip location information whenever the tip of the probe 309 is in contact with a structure of the proxy carrier 301 (e.g., as determined by a change in electrical conduction at the tip of the probe 309 when in contact with the structures having certain electrical conducting properties), in response to activation of a retractable tip that senses impact of the tip with a surface, in response to an elapsed time that the tip is in contact with a structure of the proxy carrier 301, in response to a user input (e.g., pressing a button on the probe 309), or the like. The probe 309 may be configured to determine and/or transmit tip location information continuously or periodically. In some embodiments, the probe 309 may be configured to determine and/or transmit tip location when the tip of the probe 309 is not in contact with the proxy carrier 301 or structures thereof.

The probe 309 may be in communication with, or comprised as part of, an implant placement system. The implant placement system may receive information communicated from the probe 309 such as sensor tip information, which may indicate three-dimensional coordinates of the probe tip 310 with reference to a fixed origin point within the treatment area, and may be used to calculate placement information from the carrier 301, such as in combination with coordinates of the other markings of the proxy carrier 301.

The fixed origin point (or “reference position”) may be a location on the patient, the patient bed, or elsewhere in the room. For example, a surgeon may initially touch the probe to a fixed origin point at a topmost incision point of the surgical cavity such that three-dimensional coordinates of the probe to 310 are referential to that topmost incision point of the surgical cavity. Any other fixed origin point may be used, such as a well-known anatomical feature that may be automatically (and/or manually) identified in images by the system. In this way, respective positions of each of the carriers may be determined with reference to a same fixed origin point. For example, placement information of each carrier may include an x, y, and z value indicating a three-dimensional offset from a fixed origin point. Thus, placement information for a carrier may include multiple sets of coordinates, such as three sets of x, y, z values for three different positions (e.g., corners) of the carrier.

In some embodiments, the system automatically determines an optimal fixed origin point, such as based on analysis of image or video data for an easily identifiable anatomical structure, and instructs the surgeon to calibrate the probe by touching the optimal fixed origin point.

Based on proxy carrier 301 placement information, the implant placement system may determine and/or update an expected dosimetric distribution. The expected dosimetric distribution may indicate a magnitude and/or direction of radiation expected to be emitted from a hot carrier placed at a same or similar location or orientation as the proxy carrier 301, in combination with the expected radiation to be emitted from the already implanted hot carriers 304A, 304B, 304C. The expected dosimetric distribution may be displayed for viewing by a medical professional to aid in real-time (e.g., during the medical procedure) placement of the proxy carrier 301 and/or subsequent carriers.

FIG. 3B illustrates an example tumor bed with carriers 304 (e.g., carriers 304A, 304B, 304C, 304D, 304E, 304F) placed therein. The carriers 304 may have been placed in the tumor bed subsequent to a proxy carrier having been placed in the tumor bed, for example, as shown and discussed with reference to FIG. 3A. The carriers 304 may have been placed in the tumor bed in a same or similar location or orientation as a previously placed proxy carrier. The carriers 304 may have been placed in the tumor bed to match or satisfy an expected dosimetric distribution and/or dosimetric intent, such as determined by an implant placement system and based at least on the placement information of a previously placed proxy carrier. Advantageously, placing the carriers 304 in the tumor bed according to real-time feedback provided by a dose feedback system (e.g., proxy carriers and implant placement system) may improve the radiation dosage delivered in the tumor bed or the radiation dose delivered to the patient in the tumor bed.

### Example Feedback Devices and Methods

FIGS. 4A-4F illustrate example planning images 400 (e.g., planning images 400A, 400B, 400C, 400D, 400E, 400F) displayed via a feedback device of a dose feedback system. Advantageously, the planning image 400 may be used (e.g., viewed) by medical professionals, such as surgeons or radiation oncologists in an operating room during a medical procedure to enhance a visualization of an expected dosimetric distribution and carrier placement within a tissue site of a patient. In some embodiments, the planning image 400 may be displayed on a screen such as a computer monitor, a television monitor, a laptop screen, a handheld device, a mobile device such as a smartphone, or the like. In some embodiments, the planning image 400 may be displayed within a virtual reality (VR) or augmented reality (AR) system, such as on a VR or AR headset. In some embodiments, the planning image 400 may be displayed as 3 dimensional. For example, expected dosimetric distributions may be displayed as 3 dimensional. In some embodiments, the planning image 400 may be interactive. For example, the planning image 400 may include a touch screen configured to receive user input via a user touch on the planning image 400.

As shown in the examples provided herein, the planning image 400 may display one or more live videos or images simultaneously. For example, the planning image 400 may display one or more videos or images as superimposed, merged, or overlaid with an expected dosimetric distribution and/or other information or images. As another example, after removal of proxy carriers from a surgical site, the planning image 400 may display an image of carriers superimposed on a video of a surgical site at locations where the proxy carriers were removed from and, correspondingly, the hot carriers should be placed.

In some embodiments, the planning image 400 may display a video of a surgical site in real-time as the video data is captured by one or more imaging devices. In some embodiments, the planning image 400 may display a video or image of a surgical site that is adjusted (e.g., by deformation algorithms) to account for deformation that may have occurred to the surgical site during the medical procedure. In some embodiments, the video or image may be adjusted to account for deformation dynamically, continuously, periodically, and/or in real-time as the medical procedure occurs.

FIG. 4A illustrates an example planning image 400A of a feedback device of a dose feedback system. The planning image 400A displays a video or image of a surgical site, such as a cranial tumor bed of a patient. The planning image 400A displays an expected dosimetric distribution including isodose lines 407A, 407B and 407C superimposed on a live image of a patient treatment surface where a proxy carrier 401 and a hot carrier 404A are currently positioned. The overlaid dosimetric information also includes a sensitive area indication 411, which may correspond to sensitive tissue areas, such as nerves or other organs, or to normal tissue in some embodiments.

The isodose lines 407A, 407B, 407C define ranges of radiation doses that will occur at certain locations as a result of placing a hot carrier at the location of the proxy carrier 401, in combination with the radiation from already-placed hot carrier 404A. The expected dosimetric distribution may be based on the placement information and carrier characteristics of the proxy carrier 401 and hot carrier 404.

In the example of FIG. 4A, the isodose lines 407A-407C define isodose regions therebetween associated with different radiation dose ranges and which are distinguished from one another by various visual features, such as different colors, shadings, patterns, transparencies, etc. In the illustrated example, the denser dot patterns indicate higher radiation doses. In some embodiments, the isodose regions may not include variations in shading, but may only include concentric ovular, circular, or non-circular closed loops to indicate changes in radiation dose. In some embodiments, the isodose regions may include radiation dose numbers to show an expected radiation dose at that location (e.g., within closed loops of the expected dosimetric distribution).

The isodose regions may be semi-transparent so that objects within a same physical space may be viewed simultaneously. For example, the underlying tissue and one or more carries may be viewed simultaneous with the expected dosimetric distribution depicted in FIG. 4A. In some embodiments, an expected dosimetric distributions can be displayed or not displayed within the planning image 400A, such as in response to a user selection. For example, a user may select to toggle display of the isodose lines 407A, 407B, 407C and the corresponding shading of the isodose regions, on or off as desired.

As discussed above, the dose feedback system may determine the placement information of the proxy carrier 401 through the use of a probe 309. For example, three-dimensional location information (e.g., distance from a fixed origin point in each of three dimensions) of the probe 309 tip when in contact with each of the reference markings on the proxy carrier 401 may be used to determine placement information (e.g., precise location and orientation within a treatment surface of the patient). In some embodiments, the dose feedback system may use any of the techniques, devices, or systems described herein, such as cameras, lasers, other sensors, and/or communication with the proxy carrier 401, to determine the proxy carrier 401 placement. For example, in some embodiments one or more imaging devices may be used to determine location information of dumb carriers.

In embodiments wherein the proxy carrier 401A is smart proxy carrier, placement information of the proxy carrier 401A maybe be determined internally and transmitted to the implant placement system. In such an embodiment, the expected dosimetric distribution may be updated in real-time when the proxy carrier 401A moves (e.g., within the planning image 400A and/or relative to the other features or objects displayed in the planning image 400A).

In some embodiments, the expected dosimetric distribution may not be updated with every movement of the proxy carrier 401, but may be updated as soon as the dose feedback system updates the location of the proxy carrier 401 (e.g., after the tip of the probe 309 is touched to the proxy carrier 401). The expected dosimetric distribution may continue to be displayed in the planning image 400A even after the proxy carrier 401 is removed from the surgical site and is no longer within the view of the planning image 400A. After the proxy carrier 401 is removed from the surgical site, a hot carrier may be placed at a location and orientation within the surgical site to match the location and orientation of the previously placed proxy carrier 401 so that the expected dosimetric distribution is similar or identical to an actual radiation dose emitted from the newly placed carrier.

In this example, the planning image 400A displays a sensitive area 411. The sensitive area indicator 411 (also referred to as simply “sensitive area 411”) identifies a portion of tissue that is sensitive to radiation such as a nerve or brain tissue, and which a medical professional may desire to avoid exposing to radiation. The sensitive area indicator 411 may be overlaid on locations of a planning image that were identified manually or automatically (e.g., by an implant planning system) through analysis of pre-operative or intra-operative images (e.g., CT, MRI, tractography, or connectome imaging), optionally with deformation algorithms applied.

In this example, the sensitive area 411 is displayed via the planning image 400A as shaded. In some embodiments, the sensitive area may be displayed as colored, outlined, textured, or the like. In some embodiments, the sensitive area 411 may not be displayed via the planning image 400A differently than the surrounding tissue. In some embodiments, more than one sensitive area 411 may be displayed, for example, if there is more than one location of sensitive tissue within the view of the planning image 400A. In some embodiments, a sensitive area 411 may not be displayed, for example, if there is not a location of sensitive tissue within the view of the planning image 400A.

In some embodiments, the sensitive area 411 can be displayed or not displayed within the planning image 400A such as in response to a user selection. For example, a user may select to toggle display of the sensitive area 411 on or off as desired.

In some embodiments, various depths of the patient anatomy and the corresponding expected dosimetric distribution at those depths may be displayed. The system may indicate if the expected depth of radiation is within or beyond an acceptable limit. For example, a dosimetric plan may indicate that a radiation dose of less than 60 Gy (e.g., at least 60 Gy) is expected at depths of 5 mm and more. Thus, if the system determines that more than 60 Gy is expected at a depth of greater than 5 mm, a depth alert may be triggered to indicate to the medical professional that the expected dosimetric distribution violates the acceptable limit.

A user, such as a medical professional, can view the planning image 400A to determine whether the expected dosimetric distribution is acceptable, and/or would violate a dosimetric intent or goal. For example, a medical professional may view the planning image 400A to determine whether a radiation dose of the expected dosimetric distribution reaches an expected depth and/or whether a certain radiation dose reaches the sensitive area 411. A medical professional may view the planning image 400A to make such a determination in real-time while placing proxy carriers or hot carriers within the tumor bed of the patient such as during the medical procedure. Advantageously, the planning image 400A can provide a quick and simple indication of whether an expected dosimetric distribution is acceptable which may improve a radiation dose delivered to the patient (e.g., proper radiation doses delivered to certain tissues), may prevent post-operative imaging to determine radiation doses resulting from carrier placement, and/or may prevent subsequent medical procedures to adjust a radiation dose.

In the example planning image 400A, the expected dosimetric distribution may be acceptable. For example, the isodose lines 407A-407C indicate that an estimated desired radiation dose will be delivered to a certain area and/or depth of the tumor bed and/or that minimal or no radiation will be delivered to the sensitive area 411. The expected dosimetric distribution may be determined to be acceptable by visual inspection such as via the planning image 400A and/or automatically by a dose feedback system. In some embodiments, in response to a determination that the expected dosimetric distribution is acceptable, the planning image 400A and/or a feedback device may output an indication of such. The indication may include an auditory and/or visual signal.

FIG. 4B illustrates an example planning image 400B with the expected dosimetric distribution of FIG. 4A still overlaid, and a placement indication 403B representing the placement of the proxy carrier 401 (FIG. 4A) where the corresponding hot carrier is to be placed. The placement indication 403B may be displayed in response to a determination that the expected dosimetric distribution is acceptable and the proxy carrier 401 is removed. In some embodiments, the carrier placement indication 403B may be displayed automatically. In some embodiments, the carrier placement indication 403B may be displayed in response to a user request, for example, after a user has determined that the expected dosimetric distribution is acceptable.

The carrier placement indication 403B indicates a position and/or orientation of a proxy carrier and, when the proxy carrier location is finalized, the location of the corresponding hot carrier. The carrier placement indication 403B may be displayed while the proxy carrier is still placed within the surgical site and within the view of the planning image 400B. Advantageously, the carrier placement indication 403B may be displayed after the proxy carrier has been removed from the surgical site and from the view of the planning image 400B, such as is shown in FIG. 4B. The carrier placement indication 403B may facilitate placement of a hot carrier at a same or similar location and/or orientation as previously placed proxy carrier after the proxy carrier has been removed. In the example of FIG. 4B, the carrier placement indication 403B is shown as a dotted outline of the previously placed proxy carrier, but in other embodiments may include some other indicator to indicate a proxy carrier position and/or orientation, such as an arrow, a circle, a line, an image, or the like.

In the example of FIG. 4B, the expected dosimetric distribution is displayed after a previously placed proxy carrier has been removed and after the proxy carrier placement indication 403B has been displayed. In some embodiments, the expected dosimetric distribution may not continue to be displayed after the previously placed proxy carrier has been removed and/or after the proxy carrier placement indication 403B has been displayed.

FIG. 4C illustrates an example planning image 400C as a hot carrier 404B is being position at the location of the placement indication 403B. Using the placement indication 403B, the hot carrier 404B may be placed at a similar or identical location as the proxy carrier 401 that was used in generating the expected dosimetric distribution. The hot carrier 404B may be placed by a medical professional using placement tool 420, such as forceps. The planning image 400C may display placement of the hot carrier 404B in real-time as the hot carrier 404B is being placed. A medical professional placing the hot carrier 404B may view the planning image 400C, as they are placing the hot carrier 404B to facilitate accurate placement of the hot carrier 404B as indicated by the carrier placement indication 403B. The hot carrier 404B includes one or more radiation sources (e.g., seeds) that match the radiation characteristics of the proxy carrier 401 from which the expected dosimetric distribution was determined.

FIG. 4D illustrates an example planning image 400D wherein the expected dosimetric distribution is updated as another proxy carrier 401D is positioned on the treatment surface of the patient. In the example of FIG. 4D, prior to replacement of the proxy carrier 401 with a hot carrier (e.g., as illustrated in FIG. 4C), another proxy carrier 401D is positioned on the treatment surface. For example, if the expected dosimetric distribution indicates that an inadequate level of radiation will be delivered, an additional proxy carrier 401D may be placed to increase radiation dosage to the corresponding area. Similarly, a medical professional may place a proxy carrier such as is shown in FIG. 4D after replacement of all other proxy carriers with their corresponding hot carriers (e.g., FIG. 4C).

The expected dosimetric distribution corresponding to the newly placed proxy carrier 401D may be generated and displayed in response to registration of the proxy carrier with the dose feedback system. For example, as shown, a user may use the probe 309 to touch a tip of the probe to each of the markings on the proxy carrier 401D to cause the probe 309 to communicate location data indicating position of the tip of the probe to the dose feedback system, e.g., as an x, y, z offset from a reference position. The dose feedback system may then determine placement information of the proxy carrier 401D and a corresponding expected dosimetric distribution. The planning image 400D may display the determined expected dosimetric distribution, for example, as isodose line 407C, as shown.

In the example planning image 400D, the expected dosimetric distribution 407 is shown as overlapping, contacting, or otherwise occupying a same physical space as the sensitive area 411. This may indicate that radiation would be delivered to the sensitive 411 if a hot carrier were to be placed at a same or similar location and/or orientation as the proxy carrier 401. The magnitude of radiation that would be delivered to the sensitive area 411 may correspond to a magnitude of radiation as indicated by the expected dosimetric distribution indicated by respective isodose lines 407A, 407B, 407C. In some embodiments, an expected dosimetric distribution that overlaps with a sensitive area may violate a dosimetric intent or may not be acceptable.

In some embodiments, in response to a determination that the expected dosimetric distribution violates a dosimetric intent (e.g., unwanted radiation overlaps a sensitive tissue area), the planning image may be updating to indicate a violation, such as by overlaying a violation indicator (e.g., a red “x”) on the proxy carrier and/or providing an audible alert.

FIG. 4E illustrates an example planning image 400E with an expected dosimetric distribution based on carrier characteristic indicating a non-uniform radiation distribution. In this example, the expected dosimetric distribution 417B associated with carrier placement indication 415 extends away from an origin in less than all directions and/or in less than 360 degrees. The carrier placement indication 415 may have been generated based on a placement of a proxy carrier having carrier characteristics indicating that radiation is emitted in less than all directions and/or in less than 360 degrees, for example, as a result of directional radiation shielding. Thus, the expected dosimetric distribution 417B may be determined based on the carrier characteristics of the proxy carrier to indicate emitting radiation in only certain directions (e.g., according to the radiation shielding and/or emission characteristics of the carrier that is anticipated to be placed).

FIG. 4F illustrates an example planning image 400F with an expected dosimetric distribution and dose indicators 413A, 413B overlaid. The planning image 400F also includes a carrier placement indication 403B, a hot carrier 404A, a dose callout indicator 418, a pointer 419, and a sensitive area indication 411.

In this example, the dose indicators 413A, 413B display an estimated radiation dose delivered to a portion of tissue immediately adjacent to (e.g., behind) the respective dose indicators 413A, 413B, such as based on an expected dosimetric distribution of the hot carrier 404A and the proxy carrier 403B. For example, each estimated radiation dose may be an aggregate of all determined doses from the proxy and/or hot carriers. In this example, the estimated radiation doses are displayed numerically in gray units (Gy). In some embodiments, the estimated radiation doses can be displayed as unitless numbers, for example, on a scale of one to ten as relative to adjacent radiation doses. In some embodiments, the dose indicators 413A, 413B can be displayed with colors or shading corresponding to a magnitude of radiation in a portion of the dose indicator 413A, 413B.

The planning image 400F may be interactive. For example, the planning image 400F can include a touchscreen configured to receive a user input via the planning image 400F. In some embodiments, the planning image 400F may display the dose indicators 413A, 413B in response a user selection (e.g., touch) via the planning image 400F. A user may select any portion of the planning image 400F to cause the planning image 400F to display a corresponding dose indicator to view an estimated radiation delivered to the tissue in that portion of the planning image 400F. In some embodiments, the dose indicators 413A, 413B may be hidden (e.g., cease to display) in response a user selection (e.g., touch) via the planning image 400F. In some embodiments, the dose indicators 413A, 413B may not be displayed as a default to facilitate visibility of the surgical site.

In some embodiments, the planning image 400F may display a zoomed in view of a portion of the image in response to a user input. A zoomed in view may show a finer spatial resolution. In some embodiments, the planning image 400F may display a zoomed out view of the image including additional portions of the surgical cavity not shown in FIG. 4F, in response to another user input. A zoomed out view may show a lower spatial resolution.

In some embodiments, the dose indicators 413A, 413B are updated in response to zooming in/out. For example, as a user zooms in a view of the planning image 400F, the dose indicators 413A may remain a same size relative to the size of the planning image 400F with a same number of estimated radiation doses shown (e.g., nine radiation doses shown) and the estimated radiation doses may update depending on the finer spatial resolution resulting from the zoomed in view to display estimated radiation doses over a smaller area of the surgical site. As another example, as a user zooms in a view of the planning image 400F, the dose indicators 413A, 413B may not remain a same size relative to the size of the planning image 400F (e.g., may increase relative to the size of the planning image 400G) and a number of estimated radiation doses shown (e.g., nine) may increase to display more estimated radiation doses over the same or similar area of the surgical site and the estimated radiation doses may update depending on the finer spatial resolution resulting from the zoomed in view.

In some embodiments, the dose indicators 413A, 413B are updated in response to zooming out. For example, as a user zooms out a view of the planning image 400F, the dose indicators 413A may remain a same size relative to the size of the planning image 400F with a same number of estimated radiation doses shown (e.g., nine radiation doses shown) and the estimated radiation doses may update depending on the lower spatial resolution resulting from the zoomed out view to display estimated radiation doses over a greater area of the surgical site. As another example, as a user zooms out a view of the planning image 400F, the dose indicators 413A, 413B may not remain a same size relative to the size of the planning image 400F (e.g., may decrease relative to the size of the planning image 400G) and a number of estimated radiation doses shown (e.g., nine) may decrease to display less estimated radiation doses over the same or similar area of the surgical site and the estimated radiation doses may update depending on the lower spatial resolution resulting from the zoomed out view

The planning image 400F displays a pointer 419, which is arrow shaped in this example. In some embodiments, the pointer 419 may be any other shape or size. The pointer 419 can be used by a user to interact with the planning image 400F. For example, a user may control the pointer 419 via touch on the planning image 400F, via touchpad, gestures, or with a control device such as a mouse. In some embodiments, a user may control the pointer 419 to select portions of the planning image 400F such as to drag and move proxy carrier placement indication 403B, and the like.

The planning image 400F displays a radiation callout indicator 418 that displays information relating to the expected radiation dose at the pointer location. In this example, the callout indicator 418 displays an estimated radiation dose in Gray units (Gy) of a portion of the surgical site that is immediately adjacent to (e.g., under or behind) the pointer 419. The estimated radiation dose displayed by the callout indicator 418 may be based on expected dosimetric distributions such as is indicated by isodose lines 407A, 407B. In some embodiments, the callout indicator 418 can display other information such as a radiation dose that is desired to be delivered to a portion of the surgical site, such as a maximum radiation dose is desired to be delivered, or a minimum radiation dose that is desired to be delivered.

In this example, the callout indicator 418 is displayed adjacent to the pointer 419. The callout indicator 418 may move with the pointer 419 to always remain adjacent to the pointer 419, such as when a user moves the pointer 419. The callout indicator 418 may continuously update the information it displays. For example, as a user moves the pointer 419 to different portions of the display, the callout indicator 418 may continuously update to display an estimated radiation dose of the portion of the surgical site that is immediately adjacent to (e.g., under or behind) the pointer 419 for whatever portion of the planning image 400G the pointer 419 is in and/or as the pointer 419 moves across the planning image 400F.

In some embodiments, the planning image 400F may display or not display the callout indicator 418 such as in response to a user selection to toggle display of the callout indicator 418 on or off. For example, the planning image 400F may display the pointer 419 but not the callout indicator 418, such as in response to a user selection to turn off display of the callout indicator 418.

FIG. 4G illustrates an example planning image 400G with a grid 450 overlaid on the expected dosimetric distribution and live surgical site images. The example grid 450 includes ten rows and ten columns and 100 corresponding cells. In some embodiments, the grid 450 can display any number of rows and/or columns and corresponding cells, such may be as selected by a user. The size and/or shape of the cells of the grid 450 may change depending on the number of rows and/or columns. The leftmost column of the grid 450 displays numbers (e.g., 0-9) in the cells of the column to identify respective rows. The bottommost row of the grid 450 displays letters (e.g., A-I) in the cells of the row to identify respective columns.

The grid 450 may facilitate interaction with the display and/or performing the medical procedure, such as placing carriers in the surgical site. For example, a medical professional may view the grid 450 on the planning image 400G to know in which cells of the grid 450 a carrier may be placed. In this example, the planning image 400G displays proxy carrier placement indication 403B as being within or substantially within cells 5D, 5E, 4D, and 4E. A medical professional may know that a carrier may be placed within the surgical site to appear in the planning image 400G as being within or substantially within any of cells 5D, 5E, 4D, or 4E. Additionally, in this example, the planning image 400G displays proxy carrier placement indication 403C as being within or substantially within cells 4G and 3G. A medical professional may know that a carrier may not be placed within the surgical site to appear in the planning image 400H as being within or substantially within either of cells 4G or 3G. In some embodiments, the grid 450 may include color and/or shading such as to indicate cells in which a carrier may or may not be placed.

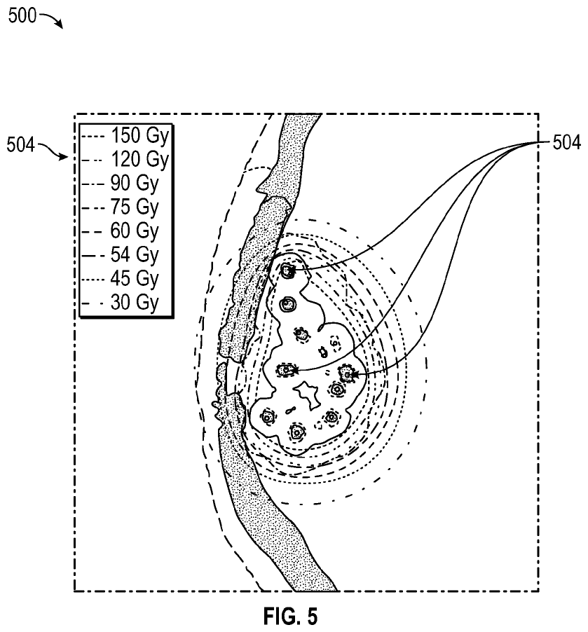

FIG. 5 illustrates an example image 500 of a postoperative surgical site with carriers 504 implanted therein. The image 500 is a computerized tomography (CT) scan. The surgical site is a cranium. The image 500 may have been taken after a medical procedure, such as within 24 hours after, to confirm carrier 504 placement and resulting radiation dose calculation and review of such as by a radiation oncologist.

The image 500 includes isodose curves indicated by different line patterns. The isodose curves correspond with and indicate various radiation doses at various tissue sites within the cranium of the patient. The legend 502 indicates radiation doses associated with the line patterns of the isodose curves. The radiation doses of the legend 502 are shown in Gray units (Gy). As shown in the example image 500, a higher radiation dose may be delivered to tissue in closer proximity to the carriers 504 than tissue in farther from the carriers 504.

### Example Dose Feedback System Implementations and Process Flows

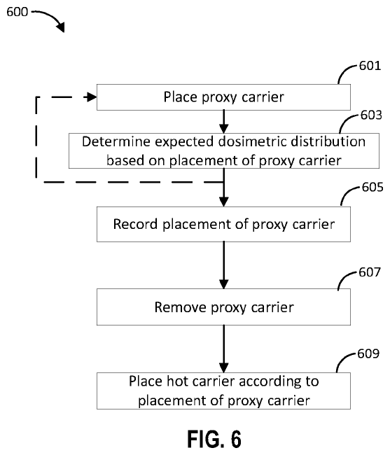

FIG. 6 is a flowchart illustrating an example process 600 for implementing a dose feedback system during intra-operative carrier placement to optimize a radiation dose delivered to a patient. The process 600, or portions thereof, can be implemented on, or executed by, a computing device of a dose feedback system such as processor 105 described with reference to FIG. 1A. The process 600 can facilitate optimizing a radiation dose delivered to a patient by providing real-time feedback (e.g., during the medical procedure) regarding the radiation doses expected to result from hot carrier placement.

At block 601, a medical professional such as a surgeon may place a proxy carrier within a surgical site of a patient, such as within a resection cavity of a tumor bed. Carrier characteristics of the proxy carrier are known (and/or may be provided and/or updated during the intraoperative carrier placement procedure), so that radiation dose and other characteristics of a corresponding hot carrier may be included in dosimetric distribution calculations. As discussed elsewhere herein, the proxy carrier placement information may be determined in several manners.

At block 603, a dose feedback system can determine the expected dosimetric distribution resulting from the placed proxy carrier. The expected dosimetric distribution can be based on placement information of the proxy carrier, as well as radiation dose information and/or other carrier characteristics.

In some implementations, block 601 and 603 may be executed repeatedly before executing any of blocks 605-609. For example, a medical professional may place multiple proxy carriers in multiple locations to update the expected dosimetric distribution before replacing the proxy carries with corresponding hot carriers.

At block 605, the dose feedback system can record placement information (e.g., a three-dimensional location and orientation) of each of the one or more proxy carriers.

At block 607, a medical professional may remove the proxy carriers from the surgical site. Proxy carriers may be removed one by one, as they are replaced by corresponding hot carriers, or proxy carriers may all be removed together and then replaced with corresponding hot carriers.

At block 609, a medical professional can place a hot carrier in the surgical site. The hot carrier can be placed according to the recorded placement of the proxy carrier that was previously removed, such as to match a position and/or orientation of the removed proxy carrier. For example, in some embodiments a carrier placement indicator may be projected onto the treatment surface showing location for each of the hot carriers and/or for a next hot carrier to be embedded on the treatment surface.

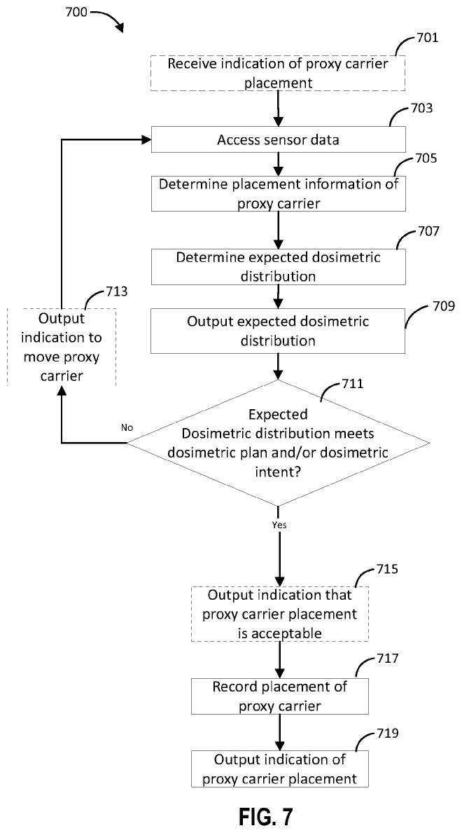

FIG. 7 is a flowchart illustrating an example process 700 for providing intra-operative dose feedback to optimize a radiation dose when placing carriers during a medical procedure. The process 700, or portions thereof, can be implemented on, or executed by, a computing device of a dose feedback system such as processor 105 described with reference to FIG. 1A.

At block 701, the dose feedback system receives an indication of placement of a proxy carrier at the treatment site. For example, a voice command or button on the probe may be pressed to indicate that a proxy carrier has been placed and a dosimetric distribution should be updated. In some embodiments, the dosimetric distribution is calculated continuously, without indication from the user that the proxy carrier has been placed.

At block 703, sensor data that is usable to determine placement information of the proxy carrier is accessed. As discussed herein, various methods for determining placement information of a particular area may be used. In an example where a dumb proxy carrier is implanted (e.g., without any electronics or internal components usable to determine location), a probe may be used to establish placement information of the proxy carrier and register the proxy carrier with the patient anatomy. Similarly, images of the proxy carrier may be acquired and analyzed (e.g., such as by analyzing shape and position of reference markings on the proxy carrier) to determine placement information of the proxy carrier. In the case of smart proxy carriers, placement information may be generated based on sensor data determined by the proxy carrier and/or information from the smart proxy carrier may be used by the implant planning system to determine the precise placement information of the smart proxy carrier. Any combination of sensors and sensor data may be used for determining placement information of a proxy carrier.

At block 705, the accessed sensor data is used to determine placement information of the proxy carrier. In some embodiments, the placement information indicates a distance of the proxy carrier from a fixed reference position, such as in each of three dimensions. For example, in some embodiments a reference position tag (e.g., an RFID tag that communicates with a probe) may be positioned at a particular position on the patient that is registered with the planning image on the implant planning system. For example, a reference position tag may be placed at a distal end of the tumor cavity and registered at that location on a planning image. In this embodiment, the probe (and/or sensors within a smart proxy sensor) may be configured to determine the distance to the reference tag that is usable to determine position of the proxy carrier in the resection cavity, for example. In some embodiments, multiple reference tags may be used to better enable three-dimensional position and orientation of proxy carriers to be determined. In some embodiments various types of sensor data, such as RF distance data and live video images of the tumor cavity, may be combined to better estimate position of proxy sensors on a treatment surface of the patient.

At block 707, the dose feedback system can determine an expected dosimetric distribution based on carrier characteristics associated with the proxy carrier, especially radiation dose of the hot carrier that will replace the proxy carrier.

At block 709, the dose feedback system can output the determined expected dosimetric distribution or indication thereof. For example, the dose feedback system may output a visual representation of the expected dosimetric distribution via a display screen.

At block 711, the dose feedback system can determine whether the expected dosimetric distribution is acceptable. For example, the dose feedback system can determine whether the expected dosimetric distribution matches a dosimetric plan and/or dosimetric intent that was developed pre-operatively and/or that is developed and/or updated intra-operatively. In some embodiments, the dose feedback system can determine whether the expected dosimetric distribution is acceptable by comparing with a dosimetric plan. In some embodiments, the determination can compare radiation doses of the expected dosimetric distribution within one or more threshold tolerance, such as a minimum and/or maximum dosage level. In some embodiments, a medical professional may determine whether the expected dosimetric distribution is acceptable at block 711 upon a visual inspection of the expected dosimetric distribution displayed via a display. In some implementations, even if the dosimetric distribution does not fully meet the expected radiation treatment levels of a dosimetric plan, as long as the expected dosimetric distribution does provide an undesirably high radiation dose, such as to sensitive or normal tissue sites, the carrier placement may be implemented. In some embodiments, a particular therapeutic index may be required for an expected dosimetric distribution to be acceptable. In some embodiments, a medical professions may be able to override an automatically generated indication that the expected dosimetric distribution is not acceptable.

Upon a determination that the expected dosimetric distribution is not acceptable at block 711, the dose feedback system, at block 713, may optionally output an indication to move the proxy carrier, and may provide a placement indicator showing another placement of a proxy carrier that would improve the expected dosimetric distribution to more closely align with a dosimetric plan and/or dosimetric intent. The output indication could include a visual and/or auditory signal such as via a feedback device. The output indication can indicate to a medical professional that the proxy carrier should be moved to attempt to improve the radiation dose. In some embodiments, the output indication can indicate a direction and/or distance to move the proxy carrier to improve the radiation dose.

Upon a determination that the expected dosimetric distribution is acceptable at block 711, the dose feedback system, at block 715, may optionally output an indication that the proxy carrier placement is acceptable. The output indication could include a visual and/or auditory signal such as via a feedback device.

At block 717, the dose feedback system can record the placement information of the proxy carriers. At block 719, the dose feedback system can output an indication of the recorded proxy carrier placements. For example, the dose feedback system can output an outline of the proxy carriers, via a display, as superimposed on a surgical site, to indicate the placement of the proxy carriers with respect to the surgical site, even after the proxy carriers are removed.

### Additional Embodiments

As used herein, “real-time” or “substantial real-time” may refer to events (e.g., receiving, processing, transmitting, displaying etc.) that occur at the same time or substantially the same time (e.g., neglecting any small delays such as those that are imperceptible to humans such as delays arising from electrical conduction or transmission). As a non-limiting example, “real-time” may refer to events that occur within a time frame of each other that is on the order of milliseconds, seconds, tens of seconds, or minutes. In some embodiments, “real-time” may refer to events that occur at a same time as, or during, another event. For example, “real-time” may refer to events that occur during a medical procedure.

As used herein, “system,” “instrument,” “apparatus,” and “device” generally encompass both the hardware (for example, mechanical and electronic) and, in some implementations, associated software (for example, specialized computer programs for graphics control) components.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various features and processes described herein may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

The various illustrative logical blocks and modules described in connection with the embodiments disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the herein-described embodiments, the elements of which are to be understood as being among other acceptable examples. All such modifications and variations are intended to be included herein within the scope of this disclosure. The section headings used herein are merely provided to enhance readability and are not intended to limit the scope of the embodiments disclosed in a particular section to the features or elements disclosed in that section. The foregoing description details certain embodiments. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

1. A radiation dose feedback system comprising:
one or more sensors including at least a camera positioned to generate a video stream of a treatment surface of a patient;
a display device positioned in an implant placement room to be viewed by a user;
one or more hardware processors configured to execute program instructions to cause the system to,
for each of one or more carriers positioned on the treatment surface:
determine placement information of the carrier based at least on sensor data from the one or more sensors, wherein the placement information indicates a three-dimensional position and orientation of the carrier with reference to a fixed origin point; and
determine carrier characteristics associated with the carrier, the carrier characteristics indicating at least a radiation dose;
determine, based on the determined placement information and carrier characteristics of each of the one or more carriers, an expected dosimetric distribution; and
display, on the display device, the video stream of the treatment surface overlaid with the expected dosimetric distribution.

2. The system of claim 1, wherein the one or more sensors include a probe including internal sensors configured to determine a three dimensional position of a tip of the probe with reference to the fixed origin point.

3. The system of claim 2, wherein placement information of a first carrier is determined by:
touching the probe tip to each of one or more markings on the first carrier;
determining placement information of the first carrier based at least on respective position of the probe tip while the probe tip touches respective marking.

4. The system of claim 3, wherein the first carrier includes two or more markings at known locations on a surface of the first carrier.

5. The system of claim 1, wherein the placement information for each carrier includes two or more three-dimensional coordinates each indicating an x, y, and z distance from the fixed origin point.

6. The system of claim 1, wherein the sensor data used to determine location information of a first carrier includes the video stream from the camera, wherein the hardware processor is configured to detect one or more markings of the carrier within the video stream, wherein an orientation of the first carrier is determined based on one or more of a size or a shape of the one or more markings.

7. (canceled)

8. The system of claim 6, wherein a laser sensor is configured to determine a distance from the laser to each of the one or more markings of the carrier, which is usable to determine location and/or orientation of the carrier.

9. The system of claim 6, wherein an infrared sensor is configured to determine a distance from the infrared sensor to each of the one or more markings of the carrier, which is usable to determine location and/or orientation of the carrier.

10. The system of claim 6, wherein a distance to each of the one or more markings of the carrier is determined based at least on the video data.

11. The system of claim 1, wherein the carriers include one or more of the sensors and placement information of the carriers is determined based on sensor data received from the one or more carriers.

12. (canceled)

13. The system of claim 1, wherein the one or more carriers each include one or more radiation source.

14. The system of claim 1, wherein the one or more carriers are proxy carriers that do not include a radiation source, wherein the radiation dose corresponds to hot carriers represented by the one or more carriers.

15. (canceled)

16. (canceled)

17. A radiation dose feedback system comprising:
one or more sensors configured to obtain data relating to a placement position or orientation of one or more proxy carriers with respect to a treatment area of a patient;
one or more surgical cameras configured to capture image or video data of the treatment area;
a feedback device configured to display one or more images or videos; and
one or more hardware processors in communication with the one or more sensors and the feedback device, wherein the one or more hardware processors are configured to execute program instructions to cause the system to:
generate deformation data of the tumor bed of the patient, wherein the deformation data is based, at least in part, on one or more images of the tumor bed and a deformation analysis of the tumor bed;
receive information from the one or more sensors including the data relating to a placement position or orientation of the one or more proxy carriers with respect to the tumor bed;
determine an expected dosimetric distribution, wherein the expected dosimetric distribution is based, at least in part, on the data relating to a placement position or orientation of the one or more proxy carriers and the deformation data;
receive, from the one or more surgical cameras, image or video data of the tumor bed;
display, via the feedback device, one or more images or videos of the tumor bed based on the received image or video data of the tumor bed; and
display, via the feedback device, a visual representation of the expected dosimetric distribution, wherein the visual representation is displayed as superimposed on the displayed on the one or more images or videos of the tumor bed.

18. The radiation dose feedback system of claim 17, wherein the one or more hardware processors are configured to execute program instructions to cause the system to:
receive carrier characteristics associated with the one or more proxy carriers, wherein the carrier characteristics include:
an identification of the one or more proxy carriers;
a magnitude of radiation expected to emit from one or more carriers to be placed in the tumor bed; and
a direction of radiation expected to emit from the one or more carriers; and
determine the expected dosimetric distribution, based at least in part, on the carrier characteristics.

19. (canceled)

20. The radiation dose feedback system of claim 17, wherein the one or more hardware processors are configured to execute program instructions to cause the system to:
display, via the feedback device, a depth visual indicator to indicate a desired distance from the tumor bed at which a certain radiation dose is desired to be delivered; and
display, via the feedback device, a placement visual indicator to indicate the placement position or orientation of the one or more proxy carriers in the tumor bed.

21. (canceled)

22. (canceled)

23. The radiation dose feedback system of claim 17, wherein the one or more hardware processors are configured to execute program instructions to cause the system to:
determine whether the expected dosimetric distribution is acceptable based at least in part on a degree to which the expected dosimetric distribution matches a dosimetric intent; and
in response to determining, that the expected dosimetric distribution is not acceptable, output, via the feedback device, one or more auditory or visual signals that the expected dosimetric distribution is not acceptable.

24. (canceled)

25. (canceled)

26. (canceled)

27. (canceled)

28. The radiation dose feedback system of claim 17, wherein the one or more hardware processors are configured to execute program instructions to cause the system to:
display, via the feedback device, the one or more images or videos of the tumor bed as three dimensional;
display, via the feedback device, the visual representation of the expected dosimetric distribution as three dimensional, wherein the visual representation is displayed as within the three dimensions of the one or more images or videos of the tumor bed.

29. The radiation dose feedback system of claim 17, wherein the one or more sensors is a probe comprising a tip portion, wherein the probe is configured to:
contact the one or more proxy carriers via the tip portion;
generate the data relating to the placement position or orientation of the one or more proxy carriers in response to contacting the one or more proxy carriers via the tip portion; and
transmit, to one or more hardware processors, the data relating to the placement position or orientation of the one or more proxy carriers.

30. The radiation dose feedback system of claim 17, wherein the one or more sensors is comprised as part of the one or more proxy carriers.
